(19) United States
(12) Patent Application Publication
(10) Pub. No.: US 2026/0338757 A1
(43) Pub. Date: Oct. 29, 2026

(54) SYSTEM FOR CONDITIONING A COOLING LUBRICANT EMULSION

(71) Applicant: LIQUIDTOOL SYSTEMS AG, Hasle-Rueegsau (CH)

(72) Inventor: Manfred SCHNEEBERGER, Muensingen (CH)

(21) Appl. No.: 19/116,111

(22) PCT Filed: Sep. 28, 2023
(86) PCT No.: PCT/EP2023/076873

§ 371 Date: Mar. 27, 2025

(30) Foreign Application Priority Data
Sep. 28, 2022 (EP) 22198329.9

(51) Int. Cl. G01N 33/28; B23Q 11/10; G01F 23/80; G05D 11/13

(52) U.S. Cl. CPC G01N 33/2888; B23Q 11/10; G01F 23/804; G01N 33/2847; G05D 11/138

(57) ABSTRACT

The invention relates to a system (100) for conditioning a cooling lubricant emulsion for machine tools (10) comprising:

a measurement unit (300) comprising at least one sensor for determining at least one parameter of the cooling lubricant emulsion, a provision unit (200) for providing fresh cooling lubricant emulsion, which is set up to mix a defined amount of a concentrate with a defined amount of water, and to provide the mixture as a fresh cooling lubricant emulsion, and a fill level sensor (500) which is set up to measure the fill level in a tank (60), characterized in that the provision unit (200) is spatially separated from the measurement unit (300) and is not in direct liquid exchange with the measurement unit (300), where the system (100) further comprises a distributor unit (400) which is set up to be connected to the measurement unit (300), the provision unit (200) and a tank (60), and to control the exchange of cooling lubricant emulsion between them, where the system (100) also comprises a control unit (600) which is set up to control and exchange data with the provision unit (200), the measurement unit (300), the distributor unit (400) and the fill level sensor (500).

The invention relates to a system for use in a method of determining at least one parameter and/or of conditioning a cooling lubricant emulsion for machine tools, and to the corresponding method.

### PRIOR ART

In machining of workpieces, they are given a particular shape by mechanically cutting off excess material in the form of chips with a tool. The machining methods of manufacturing include turning, milling, drilling and grinding, for example. Because of friction, the mechanical work introduced is converted here almost completely to heat.

Cooling lubricant emulsions, KSS emulsions for short, are widely used for metal machining in the metal processing industry. KSS emulsions are often produced from KSS concentrates by stirring into water. KSS concentrates generally consist of an oil component, buffer components, emulsifiers and various additives. KSS concentrates are homogeneous liquid products with oily consistency.

The cooling lubricant emulsions (KSS) are used to cool and to lubricate the machined workpiece and/or tool. For this purpose, the cooling lubricant emulsions are typically conveyed from a tank and applied to the tool or workpiece. The cooling lubricant emulsion is then collected again and returned.

During the use of the cooling lubricant emulsion, it is collected, and discharged together with chips taken from the machining process. The cooling lubricant emulsion adheres here to the chips discharged.

Before reusing the cooling lubricant emulsion, it therefore has to be freed of the chips and other contaminants. In addition, the fine spraying of the cooling lubricant emulsion at the high pressures used in the machines causes evaporation of water. This results in enrichment of the active components, such as oils, additives and the like, in the cooling lubricant emulsion.

To compensate for the discharged cooling lubricant emulsion and the evaporation of water, it is necessary to regularly replenish with low-concentration cooling lubricant emulsion, which is referred to as “topping up”. The top-up amount and concentration can be calculated from the current actual and target concentrations and the fill level difference in the tank.

GB 2547056 A discloses a system for determining the state of a liquid in a tank. The system includes one or more sensors that can each be used to determine a property of the liquid. Depending on the measured properties, a determination is made of the amount of a concentrate and/or the amount of water that has to be added in order to establish a desired state of the liquid.

U.S. Pat. No. 5,389,546 describes a continuous titration method for determining an alkalinity of a liquid used for metalworking. The method is intended to continuously take a sample of the liquid used for metalworking, to mix with a continuously increasing flow of a standard solution and to continuously determine the pH. The ratio of the two volume flow rates can be used to calculate the alkalinity. The liquid flow during the measurement is disposed of continuously during the measurement. A sensor used can alternatively be checked by feeding in a sample with known pH.

WO 2020/126257 discloses a method of determining at least one parameter of and/or of conditioning a cooling lubricant emulsion for machine tools, wherein the cooling lubricant emulsion is provided in a tank, wherein cooling lubricant emulsion is withdrawn from the tank and at least one parameter of the cooling lubricant emulsion is detected using at least one sensor. Also disclosed is an apparatus for performing the method.

### DISCLOSURE OF THE INVENTION

A system for conditioning a cooling lubricant emulsion for machine tools is proposed. The system comprises a measurement unit comprising at least one sensor for determining at least one parameter of the cooling lubricant emulsion, a provision unit for providing fresh cooling lubricant emulsion, which is set up to mix a defined amount of a concentrate with a defined amount of water, and a fill level sensor which is set up to measure the fill level in a tank.

Unlike in previously known devices, the provision unit is spatially separated from the measurement unit and is not in direct liquid exchange therewith. Instead, the system comprises a distributor unit which is set up to be connected to the measurement unit, the provision unit and a tank in which the cooling lubricant emulsion intended for conditioning is present, and to control the exchange of cooling lubricant emulsion between them. In addition, the system of the invention comprises a control unit which is set up to control the individual components of the system. What is meant by the expression “not in direct liquid exchange” is that there is no exchange of liquid between the measurement unit and the provision unit that is not effected via the distributor unit. The provision unit is preferably set up to be connected to and to be in liquid exchange with the distributor unit via a single conduit.

The control unit here is preferably set up to perform at least the following steps:
a) actuating the provision unit such that it mixes a defined amount of a concentrate with a defined amount of water and provides the mixture as fresh cooling lubricant emulsion;
b) actuating the distributor unit such that it guides fresh cooling lubricant emulsion from the provision unit into the measurement unit or into the tank, or guides cooling lubricant emulsion from the tank into the measurement unit, and/or optionally guides at least a portion of the cooling lubricant emulsion from the measurement unit into the tank;
c) receiving measurement data from the at least one sensor of the measurement unit and the fill level sensor;
d) calculating the top-up amount required to convert the fill level in the tank from a measured actual fill level to a defined target fill level;
e) calculating the top-up concentration of a fresh cooling lubricant emulsion required for a calculated top-up amount in order to convert the measured actual concentration in the tank to a defined target concentration.

During operation of the system of the invention, the components of the system are connected to one another, where the provision unit is connected to and in liquid exchange with the distributor unit, the measurement unit is connected to and in liquid exchange with the distributor unit, the distributor unit is in liquid exchange with a tank, and the control unit has a data connection with the provision unit, the measurement unit and the distributor unit. While the components of the system of the invention are connected to one another, the system of the invention can also be referred to as “device of the invention”. As long as the components of the system of the invention are not connected to one another, the system of the invention can also be referred to as a “component kit”.

According to the invention, spatial separation of two components means that the two components (for example the measurement unit and the provision unit) have no common housing, and preferably each have a housing.

While the provision unit and the measurement unit are spatially separate, the remaining components of the system may have a common housing with the provision unit, the measurement unit or with one another. For example, the measurement unit may be combined with the distributor unit in one housing. The control unit can also be combined in one housing with the provision unit, the measurement unit or the distributor unit. Alternatively, measurement unit, provision unit and distributor unit, preferably the measurement unit, provision unit, distributor unit and control unit, are spatially separated from each other.

The provision unit of the system of the invention for providing fresh cooling lubricant emulsion is set up to mix a defined amount of a concentrate with a defined amount of water, and to provide the mixture as fresh cooling lubricant emulsion.

The fresh cooling lubricant emulsion can be obtained in particular by conveying water and/or concentrate using a volumetric pump and/or by measuring a conveyed amount of water and/or concentrate and controlling a conveying pump depending on the conveyed amount measured.

Preferably, the provision unit therefore comprises at least one volumetric pump to convey the water and/or the concentrate. Alternatively or additionally, the provision unit comprises at least one controllable pump and a flowmeter assigned to the pump, with which a volume conveyed by the pump is determined, where the provision unit is set up to control the pump depending on the volume conveyed. Various combinations are conceivable here. For example, water can be conveyed using a controlled pump and the concentrate can be conveyed via a volumetric pump. Suitable volumetric pumps include peristaltic pumps, gear pumps and piston pumps.

Furthermore, it is possible that, in particular for control of the water feed, a controllable valve is used in conjunction with a flowmeter, where the valve is controlled depending on the measured flow rate. The water is preferably obtained from a pressurized conduit (e.g. tap water). In that case, the water does not have to be pumped. In that case, the flow rate of the water can also be measured without additional control. The concentrate is then adapted to the flow of water by means of a volumetric pump or with a controllable pump and a flowmeter. This allows for a simple configuration of the valve through which the water flows, since only opening and closing of the valve is required.

Suitable valves may be motorized valves, solenoid valves, pneumatic valves or hydraulic valves, for example. Solenoid valves are preferred.

It is also the case that the provision unit comprises a water inlet through which fresh water can be conveyed into the provision unit as described above. The water inlet may, for example, be a connector for a pressurized water conduit through which tap water or treated water, for example demineralized water or deionized water, can enter the provision unit. Alternatively, the water can be transported from a water vessel into the provision unit. This can be effected by means of a volumetric pump (water pump), where the water inlet is connected to a water suction conduit immersed into the water vessel. In this case, it may be the case that the system of the invention comprises a fill level sensor for determining the water level in the water vessel.

The provision unit may have at least one check valve in order to allow water flow in one direction only. This can prevent damage to some sensitive components and prevent contamination of the fresh water in the water pipe or water reservoir.

It is also the case that the provision unit comprises a concentrate inlet through which the concentrate of the cooling lubricant emulsion can be conveyed into the provision unit. This involves conveying of the concentrate from a concentrate vessel into the provision unit.

This can be effected by means of a volumetric pump (concentrate pump), where the concentrate inlet is connected to a concentrate suction conduit immersed in the concentrate vessel. It may also be the case that the provision unit additionally comprises a means of measuring the flow rate or the amount of concentrate conveyed. In addition, the provision unit may have one or more check valves to allow concentrate flow only in one direction. This can prevent damage to some sensitive components and prevent dilution of the concentrate in the concentrate vessel by the fresh water present in the provision unit. This is preferred especially because the system requires a constant concentration of the concentrate in the concentrate vessel, and dilution should therefore be avoided as far as possible.

The provision unit preferably comprises at least one check valve to allow the flow of water in one direction only, and at least one check valve to allow the flow of concentrate in one direction only. When the provision unit is set up to be connected to a pressurized conduit, a check valve is preferably disposed between the water inlet and a flowmeter. When the provision unit is set up to convey water and/or concentrate by means of a volumetric pump, a check valve is preferably disposed downstream of the volumetric pump and a flowmeter in flow direction, but upstream of the mixing point of water and concentrate.

Preferably, for provision of fresh cooling lubricant emulsion, the concentrate is conveyed out of the concentrate vessel via a concentrate suction conduit with a riser tube, where a fill level of the concentrate in the concentrate vessel is determined by introducing compressed air into the concentrate suction conduit, wherein an air pressure in the concentrate suction conduit is measured with a pressure sensor, a limiting pressure for a pressure rise is determined, and the limiting pressure is used to calculate the fill level in the concentrate reservoir.

Other suitable fill level sensors that can be used to determine the fill level include floats or ultrasound-based sensors. In addition, it is possible to utilize sensors based on TDR (time domain reflectometry) technology.

The provision unit may also include a mixer. The provision unit preferably includes a static mixer for mixing of water and concentrate. Good mixing of the conveyed concentrate with the water is achieved by means of the mixer, such that a homogeneous emulsion is provided.

It may optionally be the case that the provision unit is additionally set up to add additives to the fresh cooling lubricant emulsion in order to alter its properties as required. For this purpose, the additives can be conveyed from an additive vessel, for example, in a similar manner to the way in which the concentrate is conveyed from the concentrate vessel. In this case, the additive is preferably added to the fresh cooling lubricant emulsion in the mixer for mixing of water and concentrate.

It is also the case that the provision unit comprises an outlet through which the fresh cooling lubricant emulsion provided is conveyed out of the provision unit. For example, the outlet may be configured as a connector to a conduit, for example a pipe or hose.

The outlet is set up to be connected to the distributor unit in order to allow liquid exchange therewith.

The provision unit may additionally also include other components. For example, the provision unit can be actuated by the control unit in order to determine and/or alter the amount of water and concentrate conveyed. For this purpose, the provision unit may have at least one interface that enables communication with the control unit. This is typically connected to the components of the provision unit via an electronic circuit in order to enable control of the components. In addition, firmware is typically installed for control of the components.

Such an interface may be any interface that enables data transmission, for example an interface for a data cable via which the provision unit can be connected to the control unit, or a wireless interface that enables a wireless data connection (i.e. via electromagnetic waves) to the control unit. The provision unit preferably includes a wireless interface. Suitable wireless interfaces may be, for example, interfaces for communication via a Bluetooth® connection, a wireless local network (WLAN), a wireless personal network (WPAN), free-space optical communication, mobile communication, infrared technology, radio technology or the like. A wireless interface is preferred, for example a Bluetooth® interface, a WLAN interface or a mobile communication interface. Moreover, the measurement unit may have further interfaces, in order, for example, to be able to establish a direct connection with other components of the system, for example to the fill level sensor for determination of the fill level in the concentrate vessel.

The provision unit can likewise be supplied with power in any desired manner. For example, the provision unit may include an AC connector, a DC connector, a battery compartment, and/or a permanently installed rechargeable battery. An AC connector or DC connector is preferred since these allow continuous and automated operation without the need for a regular battery change or regular charging of the permanently installed battery. In the case of an AC connector, the provision unit may further comprise an AC/DC converter in order to be able to supply DC-powered parts of the provision unit.

The measurement unit of the system of the invention comprises at least one sensor for determining at least one parameter of a cooling lubricant emulsion. Cooling lubricant emulsion can be directed past at least one sensor in the measurement unit.

The at least one sensor is preferably selected from the group comprising pH sensors, conductivity sensors, photometers, light barriers and refractometers.

A refractometer used in connection with the device and further sensors are described, for example, in WO 2020/126257.

It is also the case that the measurement unit has an inlet through which liquids such as cooling lubricant emulsion can enter the measurement unit, and has an outlet through which the liquids can exit the measurement unit again. At least the inlet is set up here to be connected to the distributor unit of the system in order to allow exchange of liquid between the distributor unit and the measurement unit. The outlet is preferably also set up for the purpose of being connected to the distributor unit.

The measurement unit may additionally also include further components. For example, the measurement unit may be controlled by the control unit in order to implement measurement of at least one parameter of a cooling lubricant emulsion and to transmit the measurement data to the control unit. For this purpose, the measurement unit may have at least one interface that enables communication with the control unit. This is typically connected to the components of the measurement unit via an electronic circuit in order to enable control thereof. In addition, firmware is typically installed for control of the components.

Suitable and preferred interfaces have already been described for the provision unit and are also suitable or preferred for the measurement unit. The measurement unit preferably comprises a wireless interface for communication with the control unit.

Moreover, the measurement unit may have further interfaces, in order, for example, to be able to establish a direct connection with other components of the system, for example to the distributor unit.

The measurement unit can likewise be supplied with power in any desired manner. Suitable and preferred modes of power supply have already been described for the provision unit and are also suitable or preferred for the measurement unit.

The measurement unit is typically set up to receive cooling lubricant emulsion from the distributor unit, to measure at least one parameter of the cooling lubricant emulsion using the at least one sensor, to transmit the measurement data to the control unit and to deliver the cooling lubricant emulsion back into the distributor unit or directly into a tank. Preferably, the measurement unit is set up to transfer the cooling lubricant emulsion back into the distributor unit after a measurement.

The distributor unit of the system of the invention is set up to be connected to, and to control the exchange of cooling lubricant emulsion between, the measurement unit, the provision unit and a tank.

For this purpose, it may be the case that the distributor unit comprises several inlets and outlets for cooling lubricant emulsion, which may be configured, for example, as connections to conduits, such as pipes or hoses.

In particular, the distributor unit may include the following types of connections:
A) a connector set up to be connected to the outlet of the provision unit;
B) at least one connector set up to be connected to a tank for cooling lubricant emulsion;
C) a connector set up to be connected to the inlet of the measurement unit;
D) optionally a connector set up to be connected to the outlet of the measurement unit;
E) optionally one or more additional connections, for example in order to allow partial disposal of cooling lubricant emulsion.

Some of these connections can act as a one-way inlet or one-way outlet, and some can also act as a two-way inlet and outlet. The connector of the distributor unit that are set up to be connected to the inlet of the measurement unit preferably acts as a one-way outlet. The connectors of the distributor unit that are set up to be connected to the outlets of the provision unit or of the measurement unit preferably act as a one-way inlet. The connectors of the distributor unit that are set up to be connected to a tank can preferably act as one-way inlets or outlets or as two-way inlets and outlets, depending on the design of the distributor unit.

It may also be the case that the distributor unit comprises at least one volumetric pump set up to convey cooling lubricant emulsion from a tank into the distributor unit. Alternatively or additionally, the distributor unit may comprise at least one controllable pump and a flowmeter assigned to the pump, which is used to determine a volume conveyed by the pump. Alternatively, it may be the case that a volumetric pump or a controllable pump with a flowmeter assigned to the pump are present as external components and are connected to the distributor unit.

It may also be the case that the distributor unit comprises further flowmeters which, for example, measure the volume of cooling lubricant emulsion that enters the distributor unit from the provision unit and/or the measurement unit and/or is conveyed from the distributor unit into the measurement unit or a tank and/or is conducted out of the distributor unit to be disposed of.

The distributor unit of the system of the invention is preferably set up to be connected to a tank via at least one suction conduit. In this case, the suction conduit preferably comprises a solids filter, for example a chip filter, which prevents solids from the tank from getting into the suction conduit and the distributor unit.

The features of suitable pumps and flowmeters have already been described for the provision unit and are also applicable in the case of the distributor unit.

It is preferably the case that the distributor unit comprises further valves with which the flow of fresh cooling lubricant emulsion or of cooling lubricant emulsion withdrawn from a tank is controlled between the provision unit, the measurement unit and a tank.

The distributor unit may have at least one check valve to allow flow of cooling lubricant emulsion in one direction only. For example, a check valve can be used to define the function of a connector of the distributor unit as a one-way inlet or outlet. In this case, check valves are preferably disposed directly beyond the respective inlet or outlet in the distributor unit. In addition, this can prevent damage to some sensitive components and, for example, prevent contamination of the fresh cooling lubricant emulsion with the cooling lubricant emulsion withdrawn from a tank.

The distributor unit may additionally also include further components. For example, the distributor unit can be actuated by the control unit to regulate the exchange of liquid between the provision unit, the measurement unit and a tank. For this purpose, the distributor unit may have at least one interface that enables communication with the control unit. This is typically connected to the components of the distributor unit via an electronic circuit in order to enable control thereof. In addition, firmware is typically installed for control of the components. Suitable interfaces have already been described for the provision unit and are also suitable for the distributor unit.

Preferably, the distributor unit comprises an interface for a data cable, which is set up to be connected to an interface of the measurement unit via a data cable. In this case, communication with the control unit takes place via the connection to the measurement unit and via the interface of the measurement unit, which is set up to be connected to the control unit. Alternatively, the distributor unit can be connected directly to the control unit, for example via a dedicated wireless interface or via a cable.

The distributor unit can likewise be supplied with power in any desired manner. Appropriate types of power supply have already been described for the provision unit and are also suitable for the distributor unit. The distributor unit is preferably supplied via a power connection which is set up to be connected to the power supply of the measurement unit. More preferably, the power connector is incorporated into the interface of the distributor unit for a data cable, which is set up to be connected to an interface of the measurement unit, and this interface of the measurement unit and the data cable are set up to supply the distributor unit with power, preferably DC power.

The fill level sensor of the system of the invention, which is set up to measure the fill level in a tank, may be configured, for example, as a float or as an ultrasound-based sensor. In addition, it is possible to utilize sensors based on TDR (time domain reflectometry) technology. It is also possible to use fill level sensors in which the fill level is determined by introducing compressed air into a conduit leading to the tank, where an air pressure in the conduit is measured with a pressure sensor, a limiting pressure for a pressure rise is determined and the fill level in the tank is calculated using the limiting pressure. The fill level sensor is preferably an ultrasound-based sensor.

It is preferably the case that the fill level sensor is incorporated into the distributor unit, and the distributor unit is set up to be secured to a tank in order to be able to measure the fill level in the tank.

In addition, the system may include a sensor for detecting the formation of foam. This is preferably likewise incorporated into the distributor unit.

The control unit of the system of the invention may be any type of control unit that can be connected to the provision unit, the measurement unit and the distributor unit and can control that component or the components installed therein. The control unit is preferably a computer. The control unit may also comprise several interconnected devices, for example one or more computers that may be connected to one another via a network (e.g. local network, wireless local network, mobile network).

The control unit comprises interfaces via which it can be connected to and communicate with the interfaces of the provision unit, the measurement unit and the distributor unit. For example, the control unit comprises at least one wireless interface when at least one unit selected from the provision unit, the measurement unit and the distributor unit has a wireless interface, and at least one interface for a data cable when at least one unit selected from the provision unit, the measurement unit and the distributor unit has a corresponding interface.

The control unit can likewise be supplied with power in any desired manner. Suitable and preferred modes of power supply have already been described for the provision unit and are also suitable or preferred for the control unit.

Preferably, the provision unit, the measurement unit and the control unit each have a wireless interface, where the measurement unit and the distributor unit each have an interface for a data cable, where the provision unit and the measurement unit are set up to be connected to the control unit via the wireless interfaces, and where the distributor unit together with the measurement unit are set up to be connected via the interfaces for a data cable, and where the control unit is set up to communicate with the provision unit and the measurement unit via the wireless interfaces, and to communicate with the distributor unit via the wireless interface of the measurement unit and the data cable.

The liquid exchange between the provision unit and the distributor unit, between the distributor unit and the measurement unit, and between the distributor unit and a tank takes place via external conduits such as pipes or hoses.

According to the invention, the term “conduit” refers to devices that are provided for passive transport of free-flowing materials, e.g. liquids, gases, dispersions and free-flowing solids, in particular to hose conduits and pipelines. Unless explicitly stated in the respective context, “conduit” does not refer to power conduits or data conduits.

The term “internal conduit” refers to a conduit that serves to transport free-flowing materials within a component of the system (e.g. provision unit, distributor unit, measurement unit). The term “external conduit” refers to a conduit that serves to transport free-flowing materials outside a component of the system (for example between the components of the system).

External conduits may, for example, be set up to be reversibly or irreversibly connected to the provision unit, to the distributor unit and/or to the measurement unit.

What is meant by “reversibly” is that repeated non-destructive (excluding wear damage) separation and restoration of the connection is possible. What is meant by “irreversibly” is that non-destructive separation and restoration of the connection is not possible.

Irreversible connections can have the advantage that fluid-tightness of the connections is higher compared to reversible connections and wear is lower, but they at the same time have the disadvantage that separation of the connections, for example for replacement of the individual components, is possible only with a relatively high degree of difficulty. Irreversible connections can be made, for example, by welding conduits to connectors or inlets and outlets of the provision unit, the distributor unit and/or the measurement unit.

Reversible connections, by contrast, have the advantage that the connections can be repeatedly separated and restored such that, for example, individual components can be quickly replaced if required (for example for maintenance). Suitable means of establishing reversible connections are, for example, quick-release fasteners/connections, threaded connections, magnetic connections, adhesive bonds, clamps, clips, sleeves and brackets.

Quick-release fasteners are preferred here, since they are often capable of withstanding high pressures but can be quickly separated if required. At the same time, quick-release fasteners are often designed in such a way that, in the event of separation of the connection, a valve in the fastener closes such that no liquid can escape from the conduit.

It is preferably the case that the system of the invention comprises at least one conduit for fresh cooling lubricant emulsion which enables reversible connection between the provision unit and the distributor unit.

In these cases, it is preferred that the respective conduit has at least one free end, where the at least one free end has an identification feature uniquely assigned to the conduit, and where the at least one free end is set up to be reversibly connected to the provision unit, the distributor unit or both, preferably via a quick-release fastener, and where the distributor unit, the provision unit or both are set up to recognize the identification feature on establishment of a connection with the at least one free end.

The identification feature may be any identification feature that can be uniquely assigned and identified by electronic means. For example, the identification feature may be a near-field or radiofrequency identification transponder (NFC or RFID transponder), a printed code (barcode, QR code, serial number or the like), or a coded contact field. Accordingly, the provision unit and/or the distributor unit may have a means of reading the corresponding identification feature, such as an NFC or RFID reader for reading the NFC or RFID transponder, a scanner for reading the printed code, or a reader for the coded contact field. Preferably, the identification feature is an NFC or RFID transponder, preferably an RFID transponder, and the reader is an NFC or RFID reader, preferably an RFID reader, since these have the advantage that no prescribed alignment of the transponder and the reader is required to enable recognition of the identification feature, and spatial proximity of the identification feature to the reader can be sufficient.

In this case, one end of the conduit may be irreversibly connected to the provision unit and the other end may be the free end and may be set up to be reversibly connected to the distributor unit, where the distributor unit is set up to recognize the identification feature. In this case, the identification feature is also uniquely assigned to the provision unit. The distributor unit is set up here to determine establishment of a connection (e.g. quick-release fastener connection) between the free end of the conduit and the connector of the distributor unit that is provided for the purpose (for example by closing an electrical contact in the connector), to recognize the identification feature at the free end, and to transmit the data recognized to the control unit. The control unit is set up to assign the identification feature of the conduit and of the provision unit (for example by means of a stored database) and to establish a data connection to the identified provision unit and to the distributor unit.

Alternatively, one end of the conduit may be irreversibly connected to the distributor unit and the other end may be the free end and may be set up to be reversibly connected to the provision unit, where the provision unit is set up to recognize the identification feature. In this case, the identification feature is also uniquely assigned to the distributor unit. The provision unit is set up here to determine establishment of a connection (e.g. quick-release fastener connection) between the free end of the conduit and the connector of the provision unit that is provided for the purpose (for example by closing an electrical contact in the connector), to recognize the identification feature at the free end, and to transmit the data recognized to the control unit.

The control unit is set up to assign the identification feature of the conduit and of the distributor unit (for example using a stored database) and to establish a data connection to the identified distributor unit and to the provision unit.

Alternatively, both ends of the conduit may be free ends, where one free end is set up to be reversibly connected to the distributor unit and the other free end is set up to be reversibly connected to the provision unit, and where the provision unit and distributor unit are set up to recognize the identification features at the free ends. The provision unit and the distributor unit are set up here to determine establishment of a connection (e.g. quick-release fastener connection) between the free end of the conduit and the connector of the provision unit or of the distributor unit that is provided for the purpose (for example by closing an electrical contact in the connector), to recognize the identification feature at the free end, and to transmit the data recognized to the control unit. The control unit is set up to assign the identification feature recognized by the provision unit and the identification feature recognized by the distributor unit to a respective conduit (for example using a stored database), to compare the identified conduits to each other and, in the event of a match, to create a data connection to the provision unit and to the distributor unit.

In these configurations, the control unit is able to unambiguously discern whether there is a connection between the provision unit and the distributor unit through which an exchange of liquid can take place. It is likewise possible for the control unit to unambiguously recognize which distributor unit is connected to which provision unit when several distributor units and/or provision units are available for selection, and to actuate them accordingly such that the method of the invention can be performed therewith.

In addition, it may be the case that the system of the invention comprises conduits for cooling lubricant emulsion that enable reversible connections between the measurement unit and the distributor unit and/or between the distributor unit and a tank. In these cases, the reversible connections may be set up in the same way as described above for the reversible connection between the provision unit and the distributor unit.

The system of the invention may also have been set up for the conditioning of cooling lubricant emulsion in multiple tanks for multiple machine tools. For this purpose, it is envisaged that the system comprises at least one second measurement unit, at least one second distributor unit and at least one second fill level sensor, where the second fill level sensor is set up to measure the fill level in a second tank, where the provision unit is also spatially separated from and not in direct liquid exchange with the second measurement unit, and where the second distributor unit is set up to be connected to and to control the exchange of cooling lubricant emulsion between the second measurement unit, the provision unit and the second tank, and the system is set up to condition the cooling lubricant emulsions in at least two tanks, and where the control unit is set up to determine which distributor unit the fresh cooling lubricant emulsion from the provision unit is guided through.

This has the advantage that, in the method of the invention, a provision unit can be used to condition the cooling lubricant emulsion in several tanks, which can be done in an automated process. For this purpose, each tank is connected to a separate distributor unit, and each distributor unit is connected to a separate measurement unit, but several distributor units are connected to the same provision unit.

In respect of the second distributor unit, the second measurement unit, the second fill level sensor and their connections to the provision unit and the control unit and to one another, the description of the distributor unit, the measurement unit, the fill level sensor and their connections with the provision unit and the control unit and to one another is correspondingly applicable.

The system of the invention may be set up to condition a cooling lubricant emulsion in a single tank for a machine tool. In this case, the provision unit is connected via a conduit to a single distributor unit which is in liquid exchange with a single tank. This may be viable, for example, in plants where there is only one machine tool with a single tank and no expansion to further machine tools is planned since, for example, there is no spatial capacity for further machine tools.

Alternatively, the system of the invention is set up to condition a cooling lubricant emulsion in at least two tanks for machine tools, in which case the provision unit is connected simultaneously to and is in liquid exchange with all distributor units via a branched conduit. In this case, the control unit is set up to perform the following steps:
a1) actuating the provision unit such that it mixes an amount of a concentrate defined for a selected tank of the at least two tanks with an amount of water defined for the selected tank of the at least two tanks and providing the mixture as fresh cooling lubricant emulsion;
b1) actuating the distributor unit connected to a selected tank such that it guides fresh cooling lubricant emulsion from the provision unit into the measurement unit connected to said distributor unit or into the selected tank, or guides cooling lubricant emulsion from the selected tank into the measurement unit connected to said distributor unit, and/or optionally guides at least a portion of the cooling lubricant emulsion from said measurement unit into the selected tank;
c1) receiving measurement data from the sensors of all measurement units and from all fill level sensors;
d1) calculating the top-up amount required to convert the fill level in a selected tank from a measured actual fill level to a defined target fill level;
e1) calculating the top-up concentration of a fresh cooling lubricant emulsion required for a calculated top-up amount in order to convert the measured actual concentration in a selected tank to a defined target concentration;
f1) selecting a tank in which the cooling lubricant emulsion is to be conditioned.

The tank can be selected here in an automated manner; for example, the control unit can check on the basis of received measurement data whether the fill level or concentration in all tanks is within a defined range, and select a tank when the fill level or concentration is not within the defined range.

For example, the flow of fresh cooling lubricant emulsion solely into the distributor unit connected to the selected tank can be created by opening valves in the selected distributor unit and closing them in all other distributor units. Alternatively, the system may further comprise a distribution block with valves which is built into the branched conduit between the provision unit and the distributor units and, by opening and closing the valves, determines the branch of the conduit through which the cooling lubricant emulsion is conveyed.

In this case, the distribution block is controlled by the control unit, where the data connection between the distribution block and the control unit can be established via a direct (e.g. wireless) interface, or via the interface of the provision unit, where the distribution block is connected to the provision unit via a data cable.

This may be advisable, for example, in facilities where there are several machine tools with several tanks and they are in close proximity to one another.

Alternatively, the system of the invention is set up to condition a cooling lubricant emulsion in at least two tanks for machine tools, in which case the provision unit can be connected to several distributor units via a reversible conduit, where the conduit has at least one free end, where the at least one free end has an identification feature uniquely assigned to the conduit. The at least one free end is set up to be reversibly connected to the provision unit, the distributor unit or both, preferably via a quick-release fastener, and the distributor unit, the provision unit or both are set up to recognize the identification feature when making a connection with the at least one free end.

In this case, the control unit is set up to perform the following steps:
a2) actuating the provision unit such that it mixes a defined amount of a concentrate with a defined amount of water and provides the mixture as fresh cooling lubricant emulsion, where the amount of concentrate and the amount of water are defined for the tank connected to an identified distributor unit;
b2) actuating the identified distributor unit such that it guides fresh cooling lubricant emulsion from the provision unit into the measurement unit connected to the distributor unit or into the tank connected to the distributor unit, or guides cooling lubricant emulsion from said tank into said measurement unit, and/or optionally guides at least a portion of the cooling lubricant emulsion from said measurement unit into said tank;
c2) receiving measurement data from the sensors of all measurement units and from all fill level sensors;
d2) calculating the top-up amount required to convert the fill level in the tank connected to the identified distributor unit from a measured actual fill level to a defined target fill level;
e2) calculating the top-up concentration of a fresh cooling lubricant emulsion required for a calculated top-up amount in order to convert the measured actual concentration in the tank connected to the identified distributor unit to a defined target concentration;
f2) identifying the distributor unit to which the provision unit is connected.

The tank in which the cooling lubricant emulsion is to be conditioned is selected manually here by a user. For example, the control unit is able to use received measurement data to check whether the fill level or concentration in all tanks is within a defined range and provide a warning to the user if this is not the case for any tank. The user can then be prompted to connect the provision unit to the distributor unit connected to that tank.

It is preferable here that the reversible conduit between the provision unit and the distributor unit is a hose made of a flexible but corrosion-resistant material, since this facilitates establishment and separation of the connection of the provision unit to different distributor units. This may be advisable, for example, in facilities where there are several machine tools with several tanks, but they are not in close proximity to one another. In this case, the provision unit together with a concentrate vessel and optionally a water vessel may be positioned, for example, on a mobile means of transport (for example a cart), and be moved using the means of transport between the various tanks in order to be connected to the corresponding distributor units.

Preferably, the provision unit is therefore set up to be reversibly connected to the distributor unit, and to be moved at least in a separated state independently of the measurement unit, the distributor unit and the tank.

In particular, it is advantageous here when the control unit communicates with the provision unit, the measurement units and the distributor units via wireless interfaces, since control in this case is often exerted over a great distance.

Alternatively, it is also possible for a group of distributor units that are each connected to a measurement unit and a tank to have a common conduit having a free end with an identification feature, which is set up to be reversibly connected to the provision unit. In this case, the control unit may be set up to perform steps a1) to f1) as described above on establishment of a connection to this group of distributor units.

This may be advisable, for example, in plants where there are several groups of machine tools with several tanks that are in close proximity, where the individual groups and any individual further machine tools with tanks are in different rooms.

In particular, in embodiments of the system that are set up to be connected to several tanks, it is preferable to take measures to avoid mixing of different cooling lubricant emulsions. Therefore, a compressed air conduit is preferably provided, which is connected via a compressed air valve to the conduits between the provision unit and the distributor units, or to the provision unit, such that cooling lubricant emulsion present in the conduits can be blown out. The compressed air introduced via the compressed air conduit forces any cooling lubricant emulsion present through the respective distributor unit into the respective tank. This is particularly advantageous since, as a result, chip filters present in the connection between a distributor unit and a tank, which prevent chips and particles from entering the measuring circuit, are blown out and hence cleaned. As an alternative to cleaning by compressed air, or in addition thereto, conduits can also be cleaned by using pigs.

In addition to blowing-out for cleaning purposes, the compressed air conduit can also be used to ventilate the tanks for a prolonged period of time.

In this way, especially in the case of long periods of service, it is possible to prevent the development of malodors by preventing an anaerobic state of the emulsion via the input of oxygen.

In addition, in embodiments with several tanks, the system is set up to monitor the at least one parameter of the cooling lubricant emulsion and the fill level also in the tanks that are not in liquid exchange with the provision unit.

The invention further provides a method of conditioning a cooling lubricant emulsion for machine tools, wherein a cooling lubricant emulsion is provided in a tank and wherein the cooling lubricant emulsion is withdrawn from the tank, at least one sensor is used to detect at least one parameter of the cooling lubricant emulsion, the at least one parameter is used to determine the concentration of the cooling lubricant emulsion in the tank (60), and the cooling lubricant emulsion is at least partly returned to the tank, and a fill level sensor is used to measure the fill level in the tank, a required top-up concentration and top-up amount is calculated and the fresh cooling lubricant emulsion having the calculated top-up concentration is provided in the calculated top-up amount, further comprising the step of: calibrating the at least one sensor by purging the at least one sensor and performing a measurement with fresh cooling lubricant emulsion having a defined ratio of a concentrate to water, wherein the fresh cooling lubricant emulsion is provided by a provision unit, the sensor is part of a measurement unit, the provision unit is spatially separated from the measurement unit and is not in direct liquid exchange with the measurement unit, and the provision unit, the measurement unit and the tank are connected to a distributor unit which controls exchange of cooling lubricant emulsion between them, and where the method is controlled by a control unit.

The method of the invention is preferably performed with the aid of the system of the invention as described above.

Preferably, in the method, a cooling lubricant emulsion is provided for each of at least two machine tools in a respective tank, where each of the tanks is connected to a distributor unit connected in each case to a measurement unit, where the fresh cooling lubricant emulsion is provided in a common provision unit and where exchange of the cooling lubricant emulsion between the provision unit and the distributor units is controlled manually by changing the distributor unit connected to the provision unit, or automatically by the control unit.

Except in cases where replacement is controlled manually by changing the distributor unit connected to the provision unit, the procedure is preferably fully automated.

What is meant by “fully automated” in the present context is that a user only defines the start and end of the method, and also, where appropriate, target values and tolerance ranges for the fill levels, the concentrations and/or the at least one parameter of the cooling lubricant emulsion in the tank, and only has to intervene in the method if a fault is detected.

The at least one parameter may be, for example, a refractive index, a pH, an electrical conductivity, a transmittance or a reflectivity of the cooling lubricant. In addition, combinations of at least two of these parameters can be measured within the scope of the method. The at least one parameter determined in a measurement in which one or more measurement reagents are optionally used can be used to determine further properties. Derived properties of the cooling lubricant emulsion may be selected in particular from a concentration of the cooling lubricant emulsion, a total amine/total acid value (TAm/TAc value), a buffer capacity, a metal content, an oil content and a nitrite content. In order, for example, to determine the derived properties, the method may additionally include the following step:

performing a measurement in which a sample is taken from the tank, the sample is continuously conducted past the at least one sensor in a circuit in order to detect at least one parameter of the sample, where at least one measurement reagent is added continuously or stepwise and the at least one parameter is measured continuously until the at least one parameter reaches a defined threshold and then at least a portion of the sample is returned to the tank. For example, the distributor unit may for this purpose comprise further connectors via which a measurement reagent can be added.

Especially for calculation of the top-up concentration, a reliable measurement of the concentration of the cooling lubricant emulsion in the tank is necessary. Typically, the concentration of the cooling lubricant emulsion is determined by measuring the refractive index of the cooling lubricant emulsion with a refractometer. A refractometer determines the refractive index at the interface of a transparent surface to the cooling lubricant emulsion. A major problem is the formation of oily deposits (oil films) from the oil-containing cooling lubricant emulsions on the measurement window.

These deposits cause a creeping increase in the measurement and suggest an excessively high concentration of the cooling lubricant emulsion in the tank. This in turn leads to incorrect calculation of the concentration of the fresh cooling lubricant emulsion provided for topping up, which is chosen here in particular at too low a level.

This in turn has the result that the concentration of the cooling lubricant emulsion in the tank is lower than intended, which causes problems such as reduced machining performance, increased tool wear, corrosion, and susceptibility to microbiological infestation.

Since the incorrect measurement by the refractometer occurs in a creeping manner, it is difficult to detect. It is usually the case for this purpose that a control measurement is conducted with a second refractometer. Alternatively, the measurement system can be supplied with a calibration solution that verifies the measurement accuracy of the refractometer. The calibration solutions used generally have to be removed again from the system and disposed of. A simple waste-free method by which the contamination status of the refractometer can be determined at any time would be desirable.

According to the invention, the method therefore involves purging the at least one sensor with fresh cooling lubricant emulsion and then performing a measurement of the at least one parameter of this fresh cooling lubricant emulsion using this sensor. Since the fresh cooling lubricant emulsion has a defined ratio of a concentrate to water, its properties are known, and so the measurement obtained for the at least one parameter of the cooling lubricant emulsion can be compared with a predetermined target value.

Preferably, the cooling lubricant emulsion used for calibration can be used for conditioning of the cooling lubricant emulsion accommodated in the tank rather than discarding it. Accordingly, it is preferable to direct the cooling lubricant emulsion used for calibration into the tank after the measurement.

Comparison of the measurement for the at least one parameter of the fresh cooling lubricant emulsion with the predetermined target value enables calibration of the at least one sensor. For this purpose, for example, a correction factor can be determined, with which measurements by the at least one sensor are corrected. Should no calibration be possible or a variance between the measured value and the target value greater than a defined limit, a warning message may also be issued to indicate that the sensor needs to be serviced or replaced.

For example, the refractive index of a fresh cooling lubricant emulsion with an accurately defined ratio of water to concentrate is known, and so this emulsion can be used for calibration of a refractometer. In contrast to a calibration liquid other than the cooling lubricant emulsion, the cooling lubricant emulsion can then be directed into the tank such that no waste arises. There is also no need to hold stocks of a separate calibration fluid and/or a separate cleaning fluid.

The fresh cooling lubricant emulsion provided for the calibration has a known ratio between concentrate and water. However, this ratio depends on the currently measured concentration of the cooling lubricant emulsion. The proposed calibration is preferably conducted whenever cooling lubricant emulsion has to be topped up into the tank.

Accordingly, a fresh cooling lubricant emulsion is always used for calibration, the concentration of which is known but generally has a different value for each calibration operation. Thus, the sensor can be calibrated with different cooling lubricant emulsions as test liquid, with the properties of these different cooling lubricant emulsions being known in each case. This permits distinction in particular between reduced sensitivity of the sensor and a shift in a zero point of the sensor.

Typically, the fresh cooling lubricant emulsion provided for the calibration and for the topping up of the tank contains an oil content in the range from 0% to 10%, generally with establishment of a different concentration in the fresh cooling lubricant emulsion in each top-up owing to the dependence on the concentration measured in the tank. A proportion of the oil in the range from 0% to 10% is optimal for calibration, since there is no residual oil film on the sensor at these concentrations.

In addition to refractive index, other parameters are preferably also determined in order to describe the proper state of the cooling lubricant emulsion. Particularly important parameters for this purpose are pH, buffer capacity and conductivity. Measurement electrodes are preferably used to measure pH and conductivity. Like the refractometer described above, these measurement electrodes too are prone to oiling. The measured values change in a creeping manner, which leads to undesirable incorrect measurements that are difficult to detect. Here too, it is therefore preferable to conduct purging and/or calibration using a fresh cooling lubricant emulsion with defined concentration.

The fresh cooling lubricant emulsion with the defined concentration or the defined ratio of concentrate to water is preferably obtained by conveying water and/or concentrate using a volumetric pump. Alternatively or additionally, the fresh cooling lubricant emulsion can be obtained by conveying concentrate and/or water with a controllable conveying pump, where the amount of water and/or concentrate conveyed is measured and the conveying pump is controlled depending on the measured amount conveyed.

The production of fresh cooling lubricant emulsion with a defined concentration is preferably used not only for purging and/or calibrating of the at least one sensor but whenever fresh cooling lubricant emulsion is required for topping up. Conversely, purging and calibration of the at least one sensor are preferably effected on each top-up with fresh cooling lubricant emulsion with a defined concentration.

The defined concentration is preferably determined depending on the measured concentration of the cooling lubricant emulsion stored in the tank in such a way that, on topping up with the fresh cooling lubricant emulsion with the amount required to replenish the tank, a concentration of the cooling lubricant emulsion that meets a specification is set in the tank.

The concentration can be determined here by the control unit, which firstly uses the difference between a fixed target fill level and the measured actual fill level in a tank to calculate the top-up amount required to fill the tank, and the difference between a fixed target concentration and the measured actual concentration of the cooling lubricant emulsion and the calculated required top-up amount to determine the required top-up concentration.

Particular ingredients such as oils, surface-active additives, amines, fatty acids, corrosion inhibitors, performance additives, stabilizers and/or biocides are discharged disproportionately from the cooling lubricant emulsion when chips are separated from the cooling lubricant emulsion. This leads to depletion of these ingredients in the cooling lubricant emulsion. This depletion of ingredients leads to quality losses, such as a reduction in machining performance or an increase in susceptibility to corrosion or microbial infestation.

The selective depletion of the cooling lubricant emulsion cannot be compensated for by addition of fresh cooling lubricant emulsion. It is preferably possible to use the at least one parameter of the cooling lubricant emulsion to conclude depletion of an ingredient. If such a depletion is recognized, it is preferable to selectively replace the discharged components by the addition of additives.

In this case, cooling lubricant emulsion accommodated in the tank can be conditioned by withdrawing cooling lubricant emulsion from the tank, adding at least one additive to the cooling lubricant emulsion depending on the at least one parameter, and returning the conditioned cooling lubricant emulsion to the tank. It is additionally possible to mix the cooling lubricant emulsion using a mixer before returning it to the tank.

For this purpose, for example, the distributor unit may comprise further connectors through which an additive can be added. Alternatively, the provision unit can add the additives to the fresh cooling lubricant emulsion in order to change their properties as required. For this purpose, the additives can be conveyed from one or more additive vessels, for example, in a similar manner to the way in which the concentrate is conveyed from the concentrate vessel. In this case, the additive is preferably added to the fresh cooling lubricant emulsion in the mixer for mixing of water and concentrate.

The additive is especially selected from a component of the cooling lubricant emulsion, for example of a defoamer component such as conventional polysiloxanes used for defoaming, triisobutyl phosphate, wax defoamers or mineral oil-based defoamers, a hardness-forming component, for example a calcium and/or magnesium salt such as calcium acetate, magnesium acetate, calcium sulfonate, magnesium sulfonate and solutions thereof, a corrosion-inhibiting component such as phosphoric or phosphonic esters and salts thereof, triazine derivatives and salts thereof, carboxylic acids, fatty acids, di- or polybasic carboxylic acids and neutralization products thereof, a nonferrous metal inhibition component such as triazole derivatives and salts thereof, an aluminum-inhibiting component such as silane derivatives such as tetraethyl orthosilicate or metasilicate solutions, a biocide component such as butylbenzothiazolinone, sodium omadine solution of an oil component, an amine component, a buffer component or an emulsifier.

The additives mentioned may each also be used in the form of solutions thereof in water, oil or other suitable solvents.

In addition, the provision unit can add undiluted concentrate to the cooling lubricant emulsion if it is found that the concentration of the cooling lubricant emulsion in the tank is too low and the fill level in the tank is high.

For reliable operation, it is desirable to be able to achieve early recognition that a concentrate vessel is running dry, and also any water vessel and/or any additive vessel. It is therefore preferably the case that, in the method, a measurement of a fill level of the concentrate in the concentrate vessel and optionally of the water in the water vessel and/or of the additive in the additive vessel is conducted. If the fill level falls below a defined minimum fill level, a message can be issued, for example, prompting a changeover or refilling of the respective vessel. Furthermore, it may be the case, for example, that when an empty vessel is detected, there is a shutdown or changeover to another vessel, or the respective vessel is replenished.

The method of the invention preferably comprises the following steps:
aa) providing a fresh cooling lubricant emulsion having a defined ratio of a concentrate to water in the provision unit;
bb) introducing the fresh cooling lubricant emulsion from the provision unit into the measurement unit with the aid of the distributor unit;
cc) calibrating the at least one sensor of the measurement unit with the fresh cooling lubricant emulsion directed into the measurement unit by performing a measurement with the fresh cooling lubricant emulsion having a defined ratio of a concentrate to water, with optional conducting of the fresh cooling lubricant emulsion at least partly into the tank after the measurement;
dd) withdrawing cooling lubricant emulsion from a tank and introducing the cooling lubricant emulsion into the measurement unit with the aid of the distributor unit;
ee) measuring the at least one parameter of the cooling lubricant emulsion with the at least one sensor of the measurement unit in order to ascertain the actual concentration of the cooling lubricant emulsion in the tank with reference to the at least one parameter, with optional returning of the cooling lubricant emulsion at least partly to the tank;
ff) measuring the fill level in the tank with the fill level sensor;
gg) calculating the top-up amount required to attain a defined fill level in the tank;
hh) calculating the top-up concentration required for the calculated top-up amount in order to condition the concentration of the cooling lubricant emulsion in the tank to a fixed target value;
ii) providing a fresh cooling lubricant emulsion having the calculated required top-up concentration in the calculated top-up amount with the aid of the provision unit;
jj) introducing the fresh cooling lubricant emulsion into the tank with the aid of the distributor unit in order to condition the cooling lubricant emulsion in the tank.

If the cooling lubricant emulsion is to be conditioned in several tanks, the method may also include the step of:

kk) changing the distributor unit, the measurement unit and the tank into which fresh cooling lubricant emulsion is fed from the provision unit, and repeating steps aa) to jj).

In addition, steps dd) to jj) can be repeated for each tank, such that the cooling lubricant emulsion is conditioned continuously or at regular intervals. Similarly, steps aa) to cc) can be repeated for each measurement unit, such that the calibration of the sensors is conducted at regular intervals.

Since the method of the invention is controlled by the control unit and preferably runs in an automated manner, it may especially be the case that, in the method, a target concentration of the cooling lubricant emulsion in a tank, a tolerance range for the concentration around the target concentration, a target fill level in the tank and a tolerance range for the fill level around the target fill level are fixed, and the method is conducted in an automated manner by the control unit with these fixed values.

The target concentration here is the desired concentration of the cooling lubricant emulsion in a tank. This may be found, for example, from an operating manual of a machine tool, or from datasheets of the concentrates, in which the optimal concentrations may be specified.

The target fill level is the desired fill level in the cooling lubricant emulsion tank. In general, this is a fill level where the tank is considered to be full, without the risk of escape of the cooling lubricant emulsion in the event of minor fluctuations in fill level. This may be found, for example, from an operating manual of the tank, or is marked on the tank.

The tolerance ranges are the concentration ranges and fill level ranges in which the machine tool can be operated without concern, or recommended ranges. These may be found, for example, from operating instructions for the machine tool or tank or from datasheets for the concentrates.

The target values and tolerance ranges can be defined by user input, for example by entering the values into envisaged fields in a user interface on the control unit via an input device (e.g. keyboard). Alternatively, the fixing can be automated in that the control unit reads the values from a database.

For automated operation, the control unit preferably performs the following steps in the sequence specified:
i) sending a command to the provision unit to commence providing of fresh cooling lubricant emulsion with a ratio of concentrate to water defined for a calibration, and sending a command to the distributor unit connected to the tank to commence introducing of the cooling lubricant emulsion provided by the provision unit into the measurement unit connected to the distributor unit, where the cooling lubricant emulsion from the measurement unit is optionally directed at least partly into the tank;
ii) sending a command to the measurement unit to perform a measurement of at least one parameter with the at least one sensor and to transmit the measurement data to the control unit in order to calibrate the at least one sensor;
iii) sending a command to the provision unit to stop the provision of fresh cooling lubricant emulsion and sending a command to the distributor unit to stop the introduction of the cooling lubricant emulsion provided by the provision unit into the measurement unit;
iv) sending a command to the distributor unit to commence with withdrawal of cooling lubricant emulsion from the tank and introduction thereof into the measurement unit, with returning of the cooling lubricant emulsion from the measurement unit at least partly into the tank;
v) sending a command to the measurement unit to perform a measurement of at least one parameter with the at least one sensor and to transmit the measurement data to the control unit in order to determine the actual concentration of the cooling lubricant emulsion;
vi) sending a command to the distributor unit to stop the withdrawal of the cooling lubricant emulsion from the tank and introduction thereof into the measurement unit;
vii) sending a command to the fill level sensor to measure the fill level in the tank and to transmit the measurement data to the control unit in order to determine the actual fill level in the tank;
viii) checking whether the ascertained actual fill level and the ascertained actual concentration are within the defined tolerance ranges; if so: repeat steps v) to viii);
if not: continue from step ix);
ix) calculating the required top-up amount from the difference between the defined target fill level and the ascertained actual fill level, and calculating the top-up concentration required to achieve the target concentration with the calculated top-up amount based on the actual concentration;
x) sending a command to the provision unit to commence providing of fresh cooling lubricant emulsion with the calculated top-up concentration, and sending a command to the distributor unit to commence introducing of the cooling lubricant emulsion provided by the provision unit into the tank in order to condition the cooling lubricant emulsion in the tank;
xi) when the target fill level has been attained or the calculated top-up amount has been provided, sending a command to the provision unit to stop the provision of fresh cooling lubricant emulsion and sending a command to the distributor unit to stop the introduction of the cooling lubricant emulsion provided by the provision unit into the tank.

Steps iv) to xi) are preferably repeated continuously or at intervals in order to check whether the actual concentration and the actual fill level are within the respective tolerance ranges and to correct them if necessary. In addition, steps i) to iii) are preferably repeated at intervals in order to check the calibration of the at least one sensor and to correct it if necessary. The steps are preferably repeated until a user input to end the procedure.

Moreover, the control unit is preferably additionally able to perform the following steps, which can be performed independently of the sequence specified above:
xii) checking whether the commands sent are executed correctly and issuing a warning message if at least one command is not executed;
xiii) checking that the fill level in the concentrate vessel and, if appropriate, the water vessel and/or an additive vessel is sufficient and issuing a warning message if the respective vessel is at a low fill level and needs to be exchanged, or switching the provision unit to another vessel;
xiv) checking whether a foam sensor detects significant foam formation and, if necessary, issuing a warning signal that foam should be skimmed off, or actuating the provision unit or distributor unit to dose a defoamer into the tank;
xv) storing and documenting concentrations, temperatures, fill levels, foaming, water consumption, concentrate consumption, replenishment rates, control history and/or other data.

In embodiments in which the connection between the provision unit and the distributor unit is reversible, the control unit may perform the following additional steps among others:
xvi) checking whether the reversible connection is established and issuing a warning message if the reversible connection is severed;
xvii) on establishment of a connection with a conduit having a free end with an identification feature, sending a command to the provision unit, distributor unit or measurement unit connected to the conduit to read the identification feature and identify the conduit, and assigning several units connected to a conduit as part of the same system;
xvii) if there are multiple tanks, checking that all fill levels and concentrations are within the respective tolerance range, and issuing a warning if a value is outside the tolerance range.

In embodiments in which several distributor units are connected to one provision unit, the control unit may perform the following additional steps among others:

xix) checking whether the valves of the unactuated distributor units are closed and, if necessary, sending a command to these distributor units to close the corresponding valves;

xx) when changing the actuated distributor unit, actuating a compressed air valve such that cooling lubricant emulsion from a previous operation is blown out of the conduit.

### Advantages of the Invention

Typically, in systems for monitoring cooling lubricant emulsions, the sensors have to be deinstalled for calibration, or a calibration fluid other than the cooling lubricant emulsion has to be supplied to the system, with which the measurement accuracy of the sensors is verified. In general, such a calibration liquid has to be discharged from the system and disposed of, since it can adversely affect the properties of the cooling lubricant emulsion.

The calibration using a fresh cooling lubricant emulsion, as proposed for the method of the invention, by contrast, constitutes a simple, waste-free method by which the contamination state of the sensors can be determined at any time. Furthermore, a fresh cooling lubricant emulsion with defined properties is provided, which can be used for calibration of the at least one sensor. Here too, the use of reference emulsions or calibration fluids is advantageously avoided, such that no waste arises.

The proposed system enables easy and safe handling of cooling lubricant emulsions, and it is possible to monitor the condition of a cooling lubricant emulsion accommodated in a tank and to condition it if necessary by adding additives. In particular, calibration and conditioning can be automated. This can lead to high time and cost savings, and consumption of the cooling lubricant emulsion can be significantly lowered.

Spatial separation of the provision unit and the measurement unit, and the use of a distributor unit in order to regulate the exchange of liquid between them and with a tank, and also a control unit in order to control the components, enable significantly higher flexibility in the construction of the system and in the performance of the method than is the case in known methods. In particular, the use of the system of the invention enables operation in which several machine tools with several tanks can be operated using a provision unit, even if the tanks are not in spatial proximity to each other. Furthermore, in the case of maintenance, the individual units of the system of the invention can be exchanged for other units of the same type without any need for prolonged interruption of operation of a machine tool for the period of maintenance.

In addition, the automated method allows a distinctly higher top-up frequency, which allows the concentration in the tank to be kept essentially constant.

### BRIEF DESCRIPTION OF THE FIGURES

Working examples of the invention are shown in the drawings and elucidated in detail in the description that follows. The figures show:

FIG. 1 a schematic diagram of an embodiment of a provision unit of the system of the invention,

FIG. 2 a schematic diagram of a further embodiment of a provision unit of the system of the invention,

FIG. 3 a schematic diagram of an embodiment of a measurement unit of the system of the invention,

FIG. 4 a schematic diagram of an embodiment of a distributor unit of the system of the invention,

FIG. 5 a schematic diagram of a further embodiment of a distributor unit of the system of the invention,

FIG. 6 a schematic diagram of an embodiment of the system of the invention having a tank and a concentrate vessel, where the components of the system are connected to one another by conduits,

FIG. 7 a schematic diagram of a further embodiment of the system of the invention for operating a single machine tool,

FIG. 8 a schematic diagram of a further embodiment of the system of the invention for operating multiple machine tools, and

FIG. 9 a schematic diagram of a further embodiment of the system of the invention for operating multiple machine tools using reversible connections,

FIG. 10 a schematic diagram of a combination of distributor unit and measurement unit of the system of the invention,

FIG. 11 a schematic diagram of a further embodiment of the system of the invention having a tank and a concentrate vessel, using a combination of distributor unit and measurement unit.

The description that follows of the working examples of the invention identifies identical or similar components with the same reference numerals, without repeated description of these in individual cases. The figures are a merely schematic representation of the subject matter of the invention.

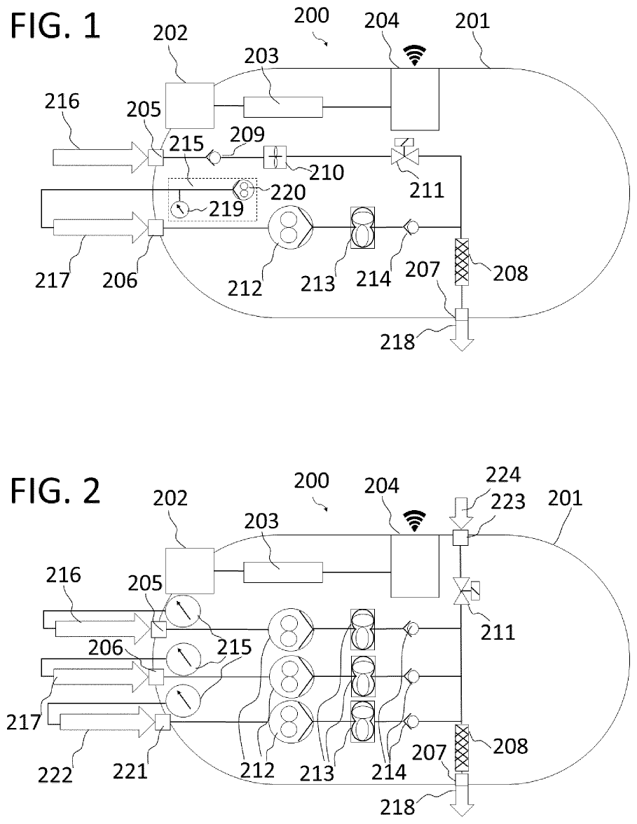

FIG. 1 shows a schematic diagram of an embodiment of a provision unit 200 for providing fresh cooling lubricant emulsion, which is set up to mix a defined amount of a concentrate with a defined amount of water, and to provide the mixture as fresh cooling lubricant emulsion.

The provision unit 200 comprises a housing 201, an AC power connector 202 which is set up to supply the provision unit with power, an AC-DC converter 203, in order to supply the DC-powered components of the provision unit with DC power, and an interface 204 set up as a wireless interface for data transmission between the provision unit 200 and a control unit. The interface 204 is connected to the electrically powered components of the provision unit via an electronic circuit (not shown) in order to allow control thereof. Furthermore, the provision unit 200 comprises a fresh water connection 205 as water inlet, which can be connected to a fresh water conduit 216, and a concentrate connection 206 as concentrate inlet, which can be connected to a concentrate vessel via a conduit 217.

Furthermore, the provision unit 200 comprises an outlet 207 for fresh cooling lubricant emulsion, to which an external conduit 218 for fresh cooling lubricant emulsion can be connected. An internal conduit leads from each of the fresh water connection 205 and the concentrate connection 206 into a static mixer 208, in which fresh water passed through can be mixed with concentrate passed through to form fresh cooling lubricant emulsion. An internal conduit continues from the static mixer 208 to the outlet 207 for fresh cooling lubricant emulsion.

The internal conduit, which proceeds from the fresh water connection 205, contains a check valve 209 which is connected downstream of fresh water connection 205 and allows the flow of fresh water only in the direction of outlet 207, in order to as far as possible avoid contamination of the fresh water by cooling lubricant emulsion. Furthermore, this internal conduit contains a flowmeter, configured as turbine 210 in this embodiment. When fresh water flows through, the turbine generates a measurable electrical current that can be converted by a control unit to a flow rate of the water flowing through the turbine. Beyond the turbine 210, this internal conduit contains an electronically controllable valve 211, e.g. a solenoid valve, with which the flow rate of the fresh water can be controlled. Such a configuration of the conduit is particularly viable when the fresh water comes from a pressurized fresh water conduit, since no active conveying of the fresh water is required here, and instead the flow velocity can be controlled by throttling via the controllable valve 211.

The internal conduit proceeding from the concentrate connection 206 contains a volumetric feed pump, which in this embodiment is configured as a gear pump 212, and a flowmeter downstream of the volumetric pump, which in this embodiment is designed as an oval gear flowmeter 213. In order to avoid contamination and/or dilution of the concentrate in a concentrate vessel by fresh water, a check valve 214 is connected downstream of the flowmeter, which allows flow only in the direction of the mixer 208. Furthermore, the provision unit 200 comprises a fill level sensor 215 for determining the fill level in a concentrate vessel. The fill level in the concentrate vessel can be determined by introducing compressed air into the concentrate conduit 217, where an air pressure in the concentrate conduit 217 is measured with a pressure sensor 219, a limiting pressure for a pressure rise is determined, and the fill level in the concentrate vessel is calculated using the limiting pressure.

In this embodiment, the fill level sensor 215 comprises, in addition to the pressure sensor 219, a volumetric air pump 220 for generating the compressed air for introduction into the concentrate conduit 217. Alternatively, the provision unit 200 may include a compressed air connection for this purpose. For example, the method for determining the fill level in the concentrate vessel is described in detail in WO 2020/126257.

For provision of the fresh cooling lubricant emulsion with this embodiment of the provision unit 200, the concentrate connection 206 and the fill level sensor 215 are connected via the concentrate conduit 217 to a concentrate vessel, and the fresh water connection 205 is connected to a pressurized fresh water conduit. The controllable valve 211 is opened in such a way that a defined amount of fresh water can flow through it. Turbine 210 is used to measure the actual amount of fresh water conveyed. If necessary, the controllable valve 211 can be used to readjust the flow rate of fresh water until the defined amount of water flows through. Depending on the flow rate of the water as measured with turbine 210, the gear pump 212 is actuated in order to convey a defined amount of concentrate out of the concentrate vessel.

The actual amount of concentrate conveyed is measured by means of the flowmeter 213 and, in the event of variances, the gear pump 212 is correspondingly actuated in order to bring the amount of concentrate conveyed into line with the defined amount.

The fill level sensor 215 measures the fill level in the concentrate vessel, and in the case of a low fill level, a signal can be sent to a control unit via the wireless interface 204 so that it can issue a warning message that the concentrate vessel should be exchanged. In the case of an empty concentrate vessel, valve 211 can be closed and the gear pump 212 can be actuated to stop the conveying of concentrate. At the same time, a signal can be sent via the wireless interface 204 to a control unit to issue an error message and, if necessary, terminate affected processes to prevent damage.

The outlet 207 for fresh cooling lubricant emulsion can be designed such that, in the absence of a connection to a conduit 218, the outlet 207 is blocked such that no leakage of cooling lubricant emulsion into the environment can take place. In this case, the flowmeters 210 and 213 do not detect any flow, even if the valve 211 is open and/or the gear pump 212 is running. In this case, a signal can be sent to a control unit to issue an error message.

FIG. 2 shows a schematic diagram of a further embodiment of a provision unit 200. This differs from the embodiment according to FIG. 1 in that the provision unit 200 is set up to convey fresh water from a fresh water vessel rather than being connected to a pressurized fresh water conduit. In this embodiment, the internal conduit leading from the fresh water connection 205 to the static mixer 208 is configured in the same way as the internal conduit leading from the concentrate connection 206 to the mixer, and hence comprises a volumetric pump, for example in the form of a gear pump 212, a flowmeter, for example in the form of an oval gear flowmeter 213, and a check valve 214 in order to prevent contamination of the water in the fresh water vessel.

Furthermore, the provision unit 200 in this embodiment comprises one or more additive connections 221 which can be connected via an additive conduit 222 to an additive vessel. An internal conduit leads from the additive connection 221 to the static mixer 208, and is configured in the same way as the internal conduits that lead from the concentrate connection and from the fresh water connection to the mixer 208. It thus comprises a volumetric pump, for example in the form of a gear pump 212, a flowmeter, for example in the form of an oval gear flowmeter 213, and a check valve 214 in order to prevent contamination of the additive in the additive vessel.

In addition, the provision unit 200 in this embodiment comprises fill level sensors 215 for the concentrate vessel, the fresh water vessel and one or more additive vessels, which can be configured in the same way as the fill level sensor 215 in

FIG. 1, and which are set up to measure the fill levels in the concentrate vessel, the fresh water vessel and the additive vessel. Furthermore, the provision unit comprises a compressed air connection 223, which can be connected to a compressed air conduit 224. An internal conduit leads from the compressed air connection 223 to the static mixer 208, and includes a controllable valve 211 which can be used to control the flow of compressed air. The internal conduit may additionally be connected to the fill level sensors 215 in order to provide the required compressed air. In this case, a pump 220, as shown in FIG. 1, is dispensable. As an alternative to the compressed air connection, the provision unit 200 may comprise a pump for generating compressed air, which is connected to the mixer 208 via an internal conduit. In this case, a check valve is provided instead of the controllable valve 211, so that the pump for generating compressed air is not damaged by cooling lubricant emulsion.

For provision of the fresh cooling lubricant emulsion with this embodiment of the provision unit 200, the concentrate connection 206 and a fill level sensor 215 are connected via the concentrate conduit 217 to a concentrate vessel, the fresh water connection 205 and a fill level sensor 215 are connected via the fresh water conduit 216 to a fresh water vessel, and the one or more additive connections 221 and a fill level sensor 215 are connected to each additive vessel.

The gear pumps 212 are actuated in order to convey a defined amount of fresh water, concentrate or additives out of the corresponding containers. The actual amounts conveyed are measured by means of the flowmeter 213 and, in the event of variances, the gear pumps 212 are correspondingly actuated in order to bring the amounts conveyed into line with the respectively defined amount. The separate conveying of additives is advantageous if individual additives from the cooling lubricant emulsion are consumed disproportionately in a machine tool, which cannot be compensated for by conveying of concentrate alone.

The conveying of fresh water and additives from corresponding vessels may be advisable in particular in embodiments of the system of the invention in which the provision unit is regularly moved between different machine tools and fresh water connections are not available everywhere.

The compressed air connection 223 can be connected to a compressed air conduit 224. This serves in particular to blow the cooling lubricant emulsion out of the internal conduit, the mixer 208 and the conduit 218 for cooling lubricant emulsion if a cooling lubricant emulsion with a different concentration is to be provided, for example when changing the machine tool for which the cooling lubricant emulsion is being provided.

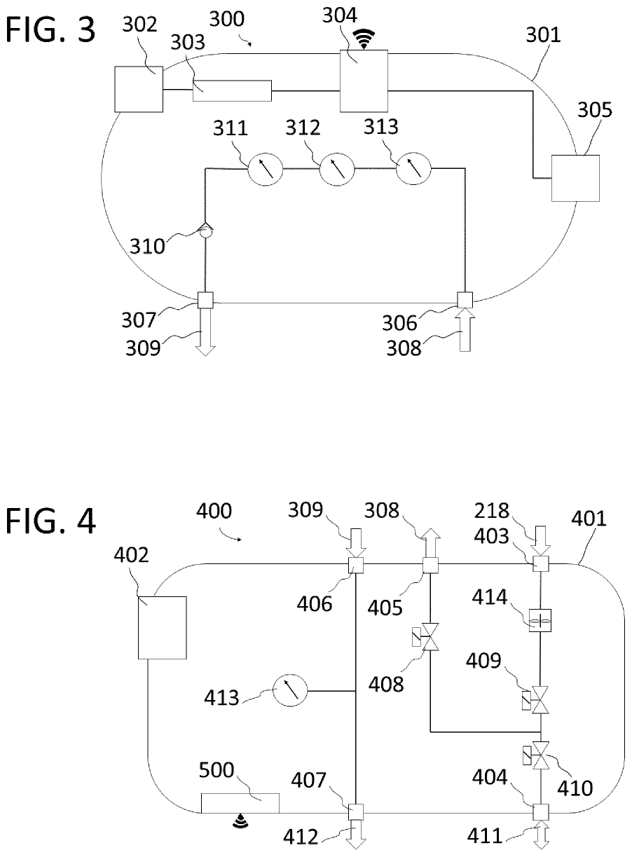

FIG. 3 shows a schematic diagram of an embodiment of a measurement unit 300 comprising sensors 311, 312 and 313 for determining at least one parameter of a cooling lubricant emulsion. The measurement unit 300 comprises a housing 301, an AC power connector 302 which is set up to supply the measurement unit 300 with power, an AC-DC converter 303, in order to supply the DC-powered components of the measurement unit with DC power, and an interface 304 set up as a wireless interface for data transmission between the measurement unit 300 and a control unit. Furthermore, the measurement unit 300 comprises an interface 305 which is set up for connection to a data cable, by means of which communication with a distributor unit is enabled.

At the same time, the interface 305 is set up to act as a DC power source for a distributor unit connected via a data cable, where the power is likewise supplied via the data cable. The interface 304 is connected to the electrically powered components of the measurement unit via an electronic circuit (not shown) in order to allow control thereof. Furthermore, the measurement unit 300 comprises a connector 306 as inlet for cooling lubricant emulsion, which can be connected to a conduit 308 for cooling lubricant emulsion, and a connector 307 as outlet for cooling lubricant emulsion, which can be connected to a conduit 309 for cooling lubricant emulsion.

Connectors 306 and 307 are connected to one another via an internal conduit. The internal conduit is connected to sensors 311, 312 and 313 for determining at least one parameter of a cooling lubricant emulsion, and is set up to direct cooling lubricant emulsion past sensors 311, 312 and 313, so that they are able to determine the at least one parameter of the cooling lubricant emulsion. For example, the sensors may be a refractometer, a pH sensor, and a conductivity sensor. In addition, the measurement unit can also include other sensors, for example photometers and light barriers. Alternatively, the measurement unit may also include only one sensor, for example only one refractometer, or two sensors.

In this embodiment, the at least one sensor 311, 312, 313 has a downstream check valve 310, which allows flow of the cooling lubricant emulsion only in the direction of the outlet connection 307. This prevents distortion of measurements owing to backflow of cooling lubricant emulsion, and the risk of damage to the sensors 311, 312, 313 is reduced.

In operation of the measurement unit 300, the inlet connection 306 is connected to a distributor unit via a conduit 308, and is supplied thereby with cooling lubricant emulsion which is to be subjected to a measurement. The outlet connection 307 may likewise be connected to the same distributor unit via a conduit 309, in order to return the cooling lubricant emulsion thereto. Alternatively, the conduit 309 can lead into a disposal vessel or directly into a tank, from which the cooling lubricant emulsion subjected to the measurement originates.

In addition, in operation, the interface 305 is connected to the distributor unit via a data cable in order to supply it with power and to enable communication of the distributor unit with a control unit via the wireless interface 304. During operation, the cooling lubricant emulsion is conducted past at the least one sensor 311, 312, 313, and at least one parameter of the cooling lubricant emulsion is determined by sensors 311, 312, 313. The measurement data is sent to a control unit via interface 304, where it is processed further.

FIG. 4 shows a schematic diagram of an embodiment of a distributor unit 400 which is set up to be connected to and to control the exchange of cooling lubricant emulsion between a measurement unit, a provision unit and a tank.

The distributor unit 400 comprises a housing 401, and an interface 402 which is set up to be connected to a measurement unit via a data cable and to communicate with a control unit via the data cable and an interface of the measurement unit. At the same time, the interface 402 is set up to supply the distributor unit with power via the data cable and via the power connection of the measurement unit. The interface 402 is connected to the electrically powered components of the distributor unit via an electronic circuit (not shown) in order to allow control thereof.

Furthermore, the distributor unit 400 comprises a connection 403 as an inlet for fresh cooling lubricant emulsion, which can be connected to a conduit 218 for fresh cooling lubricant emulsion. The conduit 218 for fresh cooling lubricant emulsion may correspond to conduit 218 as shown in FIGS. 1 and 2. In addition, the distributor unit 400 shown here comprises a two-way connection 404, which can be connected to a tank via a conduit 411, and can act both as an inlet for cooling lubricant emulsion from the tank and as an outlet for fresh cooling lubricant emulsion into the tank. In addition, the distributor unit 400 comprises a connection 405 as a further outlet for cooling lubricant emulsion, which can be connected to a measurement unit via a conduit 308. The conduit 308 may correspond to the conduit 308 as shown in FIG. 3.

Furthermore, the distributor unit 400 comprises a connector 406 as inlet for cooling lubricant emulsion that has been subjected to a measurement, which can be connected to a measurement unit via a conduit 309, and a connector 407 as outlet for this cooling lubricant emulsion, which can be connected to a tank via a conduit 412. The conduit 309 may correspond to the conduit 309 as shown in FIG. 3.

An internal conduit leads from the inlet connection 403 for fresh cooling lubricant emulsion to the two-way connection 404 with a branch to the outlet connection 405. Between the inlet connection 403 and the branch, the conduit comprises a flowmeter, which in this embodiment is designed as a turbine 414, and a valve 409, for example a solenoid valve, which is closed in the normal state and can be opened if required in order to enable flow of fresh cooling lubricant emulsion. In the branch leading to the outlet connection 405, the internal conduit contains a further valve 408, e.g. a solenoid valve, which is open in the normal state and can be closed if required. In addition, the internal conduit may comprise a further valve 410 between the branch and the two-way outlet 404, which is open in the normal state.

A further internal conduit leads from the inlet connection 406 for cooling lubricant emulsion that has been subjected to a measurement to the outlet connection 407, through which the cooling lubricant emulsion that has been subjected to a measurement can be directed into a tank. This internal conduit is connected to a pressure sensor 413.

Furthermore, the distributor unit 400 comprises the fill level sensor 500, which is configured, for example, as an ultrasound sensor and can measure the fill level in a tank. Accordingly, the distributor unit 400 is set up to be secured to such a tank in order to determine the fill level therein.

In operation, the inlet connection 403 for fresh cooling lubricant emulsion is connected via a conduit 218 to a provision unit, the two-way connection 404 is connected via a conduit 411 to a tank for cooling lubricant emulsion, the outlet connection 405 is connected via a conduit 308 to the inlet connection of a measurement unit, the inlet connection 406 is connected via a conduit 309 to an outlet connection of the measurement unit, and the outlet connection 407 is connected via a conduit 412 to the tank for cooling lubricant emulsion. In addition, the distributor unit is secured to the tank in order to be able to measure the fill level therein. Since there is no conveying pump in this embodiment for the cooling lubricant emulsion from the tank, the conduit 411 is additionally connected to a conveying pump which transports the cooling lubricant emulsion from the tank into the distributor unit 400. Alternatively, a conveying pump can be incorporated into the distributor unit 400 or be incorporated into a measurement unit connected thereto, which then draws the cooling lubricant emulsion through the distributor unit via conduit 411 and conduit 308.

In the normal state, the distributor unit 400 is set up to feed cooling lubricant emulsion from the tank for measurement into the measurement unit, since the other states (calibration and conditioning) only occur as required. Therefore, valve 409, which controls the flow of fresh cooling lubricant emulsion, is normally closed, and valves 408 and 410, which control the flow from the tank to the distributor unit and from the distributor unit to the measurement unit, are open. This ensures that only the cooling lubricant emulsion from the tank is subjected to a measurement, without the measurement being distorted by fresh cooling lubricant emulsion.

In the state of sensor calibration, valve 410, which controls the flow from the tank into the distributor unit 400, is closed, and valve 409, which controls the flow of fresh cooling lubricant emulsion, is open. If valve 410 is absent, flow from the tank into the distributor unit 400 is prevented by correspondingly actuating a corresponding conveying pump.

In the state of conditioning, valve 408, which controls the flow from the distributor unit 400 into the measurement unit, is closed, and valve 409, which controls the flow of fresh cooling lubricant emulsion, is open.

In this embodiment, the cooling lubricant emulsion which is returned from the measurement unit into the distributor unit 400 is directed through the outlet 407 and a conduit 412 connected thereto into the tank.

The pressure sensor 413 is set up here to monitor the pressure in the internal conduit between connectors 406 and 407. In the event of a sharp pressure rise (for example when conduit 412 is clogged or connector 407 is blocked since no conduit is connected) or pressure drop (for example when a conduit is severed, or conduit 309 is not connected to connector 406 or is clogged), a signal can be sent to the control unit via interface 402 for it to issue a warning message.

The fill level sensor 500 continuously measures the fill level in the tank and sends the measured data via interface 402 and an interface of the measurement unit to a control unit, where the data is processed further.

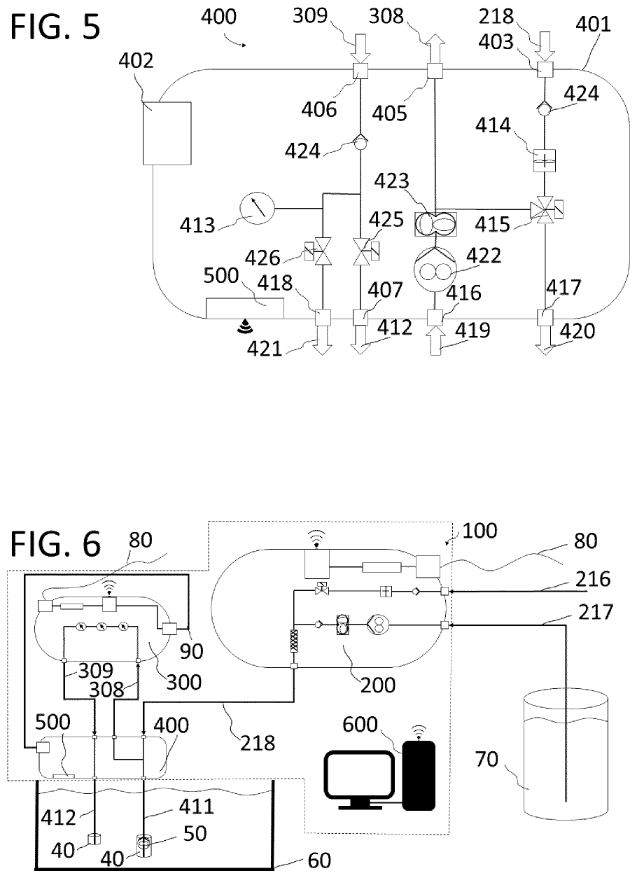

FIG. 5 shows a schematic diagram of a further embodiment of a distributor unit 400 which is set up to be connected to and to control the exchange of cooling lubricant emulsion between a measurement unit, a provision unit and a tank.

This embodiment differs from the embodiment shown in FIG. 4 in that, rather than a two-way connection 404, a one-way inlet connection 416 is provided, which can be connected to a tank via a conduit 419, and a one-way outlet 417 is provided for fresh cooling lubricant emulsion, which can be connected to the tank via a further conduit 420. Moreover, the distributor unit 400 in this embodiment comprises a further outlet 418 for cooling lubricant emulsion which has been subjected to a measurement, which can be connected to a conduit 421, discarded via the cooling lubricant emulsion or disposed of.

In this embodiment, an internal conduit leads from the inlet 403 for fresh cooling lubricant emulsion to the outlet 417 for fresh cooling lubricant emulsion, with a branch leading to the outlet connection 405. The internal conduit includes a check valve 424 to prevent flow of the fresh cooling lubricant emulsion in the wrong direction, and a flowmeter in the form of a turbine 414 in order to determine the amount of fresh cooling lubricant emulsion flowing through, both of which are between the inlet 403 for fresh cooling lubricant emulsion and the branch. Furthermore, the internal conduit comprises a three-way valve which controls the flow direction of the fresh cooling lubricant emulsion in the direction of the outlet connection 417 or the outlet connection 405. In the normal state, the three-way valve is open in the direction of outlet connection 417 and closed in the direction of outlet connection 405.

Between the inlet connection 416 for cooling lubricant emulsion from the tank and the outlet connection 405, the distributor unit 400 in this embodiment likewise comprises an internal conduit which may partly correspond to the branch of the internal conduit originating from the inlet 403. This internal conduit comprises a volumetric pump 422, which is designed as a gear pump and is set up to convey cooling lubricant emulsion from the tank through the distributor unit into the measurement unit, and a flowmeter 423 in the form of an oval gear flowmeter (optional), which is set up to verify the amount of cooling lubricant emulsion conveyed.

The internal conduit between the inlet connection 406 for cooling lubricant emulsion which has been subjected to a measurement and the outlet 407 therefor, which can be connected to the tank via a conduit 412, additionally includes a branch that leads to outlet 418, via which cooling lubricant emulsion can be disposed of. In this case, this internal conduit between the connection 406 and the branch comprises a check valve 424, which prevents flow of the already examined cooling lubricant emulsion into the measurement unit. This check valve can replace the check valve shown in FIG. 3 in the measurement unit, if one is required. Moreover, this internal conduit comprises a valve 425, for example a solenoid valve, which is open in the normal state and is between the branch and the outlet 407, and a valve 426, e.g. a solenoid valve, which is closed in the normal state and is between the branch and the outlet 418.

In this embodiment, the outlets 407 and 417 that direct cooling lubricant emulsion into the tank may be the same outlet, so that there is no need for one connection and one conduit.

The operation of this embodiment differs from the operation of the embodiment shown in FIG. 4 in that, rather than the two-way connection 404, the one-way connections 416 and 417 are connected to the tank via conduits 419 and 420, and the outlet connection 418 is connected to a conduit 421, via which cooling lubricant emulsion can be disposed of.

In the normal state, the distributor unit 400 is set up to feed cooling lubricant emulsion from the tank for measurement into the measurement unit, since the other states (calibration, disposal) only occur as required. Therefore, valve 426 which controls the disposal is closed in the normal state, and valve 425 which controls the flow from the measurement unit into the tank is open. Furthermore, the three-way valve 415 is closed in the normal state in the direction of the connection 405 which leads to the measurement unit, and open in the direction of the outlet 417 which leads to the tank. This ensures that only the cooling lubricant emulsion from the tank is subjected to a measurement, without the measurement being distorted by fresh cooling lubricant emulsion. At the same time, it is made possible for the cooling lubricant emulsion from the tank to be subjected to a measurement in the measurement unit, while the cooling lubricant emulsion in the tank is conditioned by topping up with fresh cooling lubricant emulsion. In the normal state, the cooling lubricant emulsion is continuously conveyed by the volumetric pump 422 from the tank into the distributor unit 400 and from the distributor unit 400 into the measurement unit, with monitoring of the flow by the flowmeter 423. If no flow is detected, it is possible, for example, to send a signal to the control unit to issue a warning message.

For conditioning, the distributor unit 400 remains in the normal state, and the flow of the fresh cooling lubricant emulsion can be controlled by the provision unit alone.

In the state of sensor calibration, the three-way valve 415 is opened in the direction of outlet 405 and closed in the direction of outlet 417, and the conveying pump 422 is stopped so that the calibration is not distorted by cooling lubricant emulsion from the tank.

Closure of the three-way valve 415 is envisaged in the event that the provision unit is separated from the distributor unit 400 or several distributor units 400 are connected to the provision unit.

FIG. 6 shows a schematic diagram of an embodiment of the system 100 of the invention (delimited by a dashed frame) comprising a provision unit 200, a measurement unit 300, a distributor unit 400, a fill level sensor 500 and a control unit 600, with connection of these components of the system 100 for operation.

The provision unit 200, the measurement unit 300, and the distributor unit 500 in FIG. 6 correspond to the embodiments shown in FIGS. 1, 3 and 4, the latter being depicted in a distinctly simplified manner. The connectors accordingly correspond to the connectors shown in FIGS. 1, 3 and 4, but are not given reference symbols for purposes of clarity.

The provision unit 200 is connected via a power cable 80 to an AC power source (not shown), and connected via a fresh water conduit 216 to a fresh water source (not shown). Furthermore, the provision unit is connected via a concentrate conduit 217 to a concentrate vessel 70. The provision unit 200 is connected to the distributor unit 400 via a conduit 218 for fresh cooling lubricant emulsion.

Furthermore, a wireless data connection has been established between the provision unit 200 and the control unit 600, via which the provision unit 200 and the components present therein are controlled.

The distributor unit 400 is secured to a tank 60 above the surface of the cooling lubricant emulsion and contains the fill level sensor 500 which is used to measure the fill level in the tank. Furthermore, the distributor unit is connected to a tank 60 via a suction conduit 411, where the end of the suction conduit 411 connected to the tank is immersed in the cooling lubricant emulsion, and is surrounded by a filter basket 40. Furthermore, the end of the suction conduit 411 is equipped with a liquid pump 50, with which the cooling lubricant emulsion is conveyed into the distributor unit 400 after filtering through the filter basket 40. Furthermore, the distributor unit 400 is connected to the tank 60 via a return conduit 412, through which cooling lubricant emulsion which has been subjected to a measurement is directed into the tank. The end of the return conduit 412 is likewise surrounded by a filter basket 40 in order to prevent inadvertent entry of particles into the distributor unit 400 or the measurement unit 300.

The distributor unit 400 is also connected via a conduit 308 to the measurement unit 300, via which cooling lubricant emulsion is directed into the measurement unit, and is connected via a further conduit 309 to the measurement unit 300, via which the cooling lubricant emulsion is conducted after the measurement into the distributor unit 400 and the tank 60. In addition, the distributor unit 400 is connected to the measurement unit 300 via a data cable 90, which simultaneously acts as a power cable, which assures power supply to the distributor unit 400 via the power connection of the measurement unit 300 and enables data exchange between the distributor unit 400 and the control unit 600 via the wireless interface of the measurement unit.

The measurement unit 300 is connected to a power source (not shown) via a power cable 80. The current from the power source is utilized here to supply both the measurement unit 300 and the distributor unit 400 (via the data cable 90).

Furthermore, a wireless data connection has been established between the measurement unit 300 and the control unit 600, via which the measurement unit 300 and the components present therein, and also the distributor unit 400 (via the data cable 90) and the components present therein are controlled, and measurement data are transmitted.

The control unit 600 is, for example, a computer which, via a wireless interface, can communicate with, actuate or receive information from the provision unit 200, the measurement unit 300 and the distributor unit 400 (via the interface of the measurement unit 300 and the data cable 90). Alternatively, the distributor unit 400 can have a dedicated power connection and communicate with the control unit 600 via a dedicated (e.g. wireless) interface.

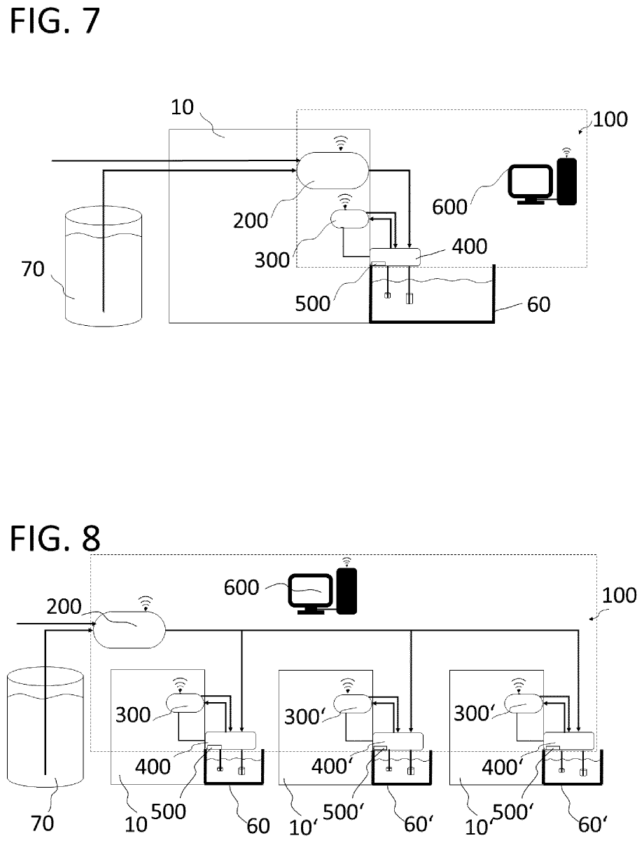

FIG. 7 shows a schematic diagram of an embodiment of the system 100 of the invention (delimited by a dashed frame) comprising a provision unit 200, a measurement unit 300, a distributor unit 400, a fill level sensor 500 and a control unit 600, with connection of these components of the system 100 for operation. In the present case, only the housings of the provision unit 200, the measurement unit 300 and the distributor unit 400 are shown. The connections essentially correspond to those in FIG. 6, which are not given reference symbols for clarity. Some of the connections are present at different sites.

The provision unit 200 and the distributor unit 300 are mounted on a machine tool 10. The distributor unit 400, as in the embodiment according to FIG. 6, is secured to a tank 60, and includes the fill level sensor 500 to measure the fill level in the tank 60. The tank 60 is connected to the machine tool 10 in order to supply it with cooling lubricant emulsion.

The provision unit 200 and the measurement unit 300 may be supplied with power from the machine tool, or connected to separate power sources via power cables.

In this embodiment, the system of the invention is set up to condition the cooling lubricant emulsion for a single machine tool 10.

FIG. 8 shows a schematic diagram of a further embodiment of the system 100 of the invention (delimited by a dashed frame). This embodiment differs from the embodiment shown in FIG. 7 in that the system 100 additionally comprises further measurement units 300′, further distributor units 400′ and further fill level sensors 500′, all of which are supplied with fresh cooling lubricant emulsion by the same provision unit 200. The system is also additionally set up to condition the cooling lubricant emulsions in further tanks 60′ for further machine tools 10′.

The provision unit 200 is also not mounted on a machine tool, and is connected to a separate power source.

The control unit controls the provision unit 200, and also all measurement units 300, 300′, all distributor units 400, 400′ and all fill level sensors 500, 500′.

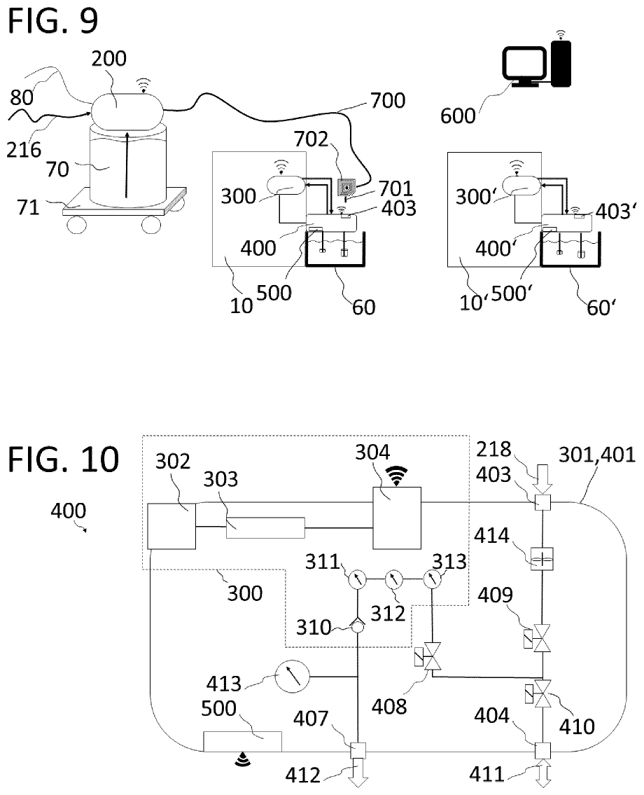

FIG. 9 shows a schematic diagram of a further embodiment of the system 100 of the invention. This embodiment differs from the embodiment shown in FIG. 8 in that the system 100 comprises a conduit 700, which is a form of the conduit 218 for fresh cooling lubricant emulsion. The conduit 700 is configured as a flexible hose and comprises a free end 701, which comprises an identification feature 702 uniquely assigned to the conduit 700 and the provision unit 200 in the form of an RFID transponder.

The free end 701 and the connection 403 or 403′ for fresh cooling lubricant emulsion in the distributor unit 400 or 400′ can be connected to one another via a quick-release fastener. The connectors 403 and 403′ are also set up to recognize the identification feature 702 in that it is connected to an RFID reader. The distributor unit 400 or 400′, on establishment of a connection to the free end 701, can read the identification feature 702 and transmit the data to the control unit 600.

The control unit 600 determines which of the distributor units 400 or 400′ has detected the identification feature 702 and has been connected to the provision unit 200 to which the identification feature 702 belongs, and controls the provision unit 200, the corresponding distributor unit 400 or 400′, the corresponding fill level sensor 500 or 500′ and the corresponding measurement unit 300 or 300′ as a coherent system for conditioning the lubricant emulsion in the tank 60 or 60′ for the machine tool 10 or 10′.

The other distributor unit, the other measurement unit and the other fill level sensor continuously conduct measurements of the at least one parameter of the cooling lubricant emulsion and the fill level in the other tank of the machine tool, and send a signal to control unit 600 if conditioning of the cooling lubricant emulsion in this tank or calibration of at least one sensor in this measurement unit is required. The control unit 600 will then issue a corresponding warning message.

Furthermore, the provision unit 200 in this embodiment is mounted on a concentrate vessel 70, which is placed on a mobile transport trolley 71, by means of which the provision unit is movable.

FIG. 10 shows a schematic diagram of one embodiment of a combination of distributor unit 400 and measurement unit 300 of the system 100 of the invention. This embodiment differs from the embodiment shown in FIG. 4 in that the distributor unit 400 and the measurement unit 300 have a common housing 301/401, and hence the components of the measurement unit 300 (delimited by a dashed frame) as shown in FIG. 3 and the components of the distributor unit 400 as shown in FIG. 4 are present within this housing.

In this embodiment according to FIG. 10, the interface 305 present within the measurement unit 300 according to FIG. 3 and the interface 402 present within the distributor unit 400 according to FIG. 4 are dispensable since power can be supplied to both units via the common AC power connector 302 and the AC-DC converter 303, and data can be transferred between the two units and a control unit, and the electrically powered components of both units can be controlled, via the common interface 304, without using an additional data cable or power cable between the two units. Likewise dispensable are the connections 306, 307, 405, 406 for liquid exchange between the measurement unit 300 and the distributor unit 400, since liquid is exchanged via internal conduits.

The use of a combination of distributor unit 400 and measurement unit 300 in a housing 301/401 enables easier handling for the end user of the system of the invention. A corresponding combination can also be created from any other embodiments of the measurement unit 300 and the distributor unit 400 in a housing.

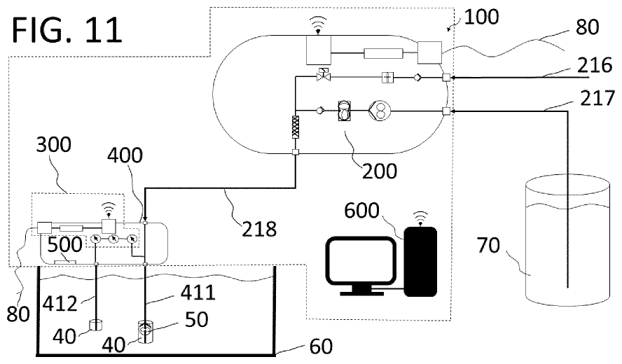

FIG. 11 shows an embodiment of the system 100 of the invention as shown in FIG. 6, except using the combination of distributor unit 400 and measurement unit 300 shown in FIG. 10 rather than the separate units. The conduits 308 and 309 and the data cable 90 are not required owing the use of this combination, which allows easier handling for the end user of the system of the invention. The combination of measurement unit 300 and distributor unit (400) in a common housing (301,401) can be used in any embodiment of the system of the invention in place of the separate units.

The invention is illustrated in detail by the examples and claims that follow.

### EXAMPLES

In the examples that follow, a cooling lubricant emulsion KSS1 is used, which is obtained by diluting a concentrate available under the Blasocut 4000 Strong name from Blaser Swisslube. Blasocut 4000 Strong is a water-miscible, mineral oil-based cooling lubricant.

### Example 1

The system 100 of the invention used is a construction as shown in FIG. 7, where the provision unit 200 is one as shown in FIG. 1, the measurement unit 300 is one as shown in FIG. 3, and the distributor unit 400 is one as shown in FIG. 4. The power connections are all connected to a power source; the provision unit 200 is also connected to a concentrate vessel and to a pressurized fresh water conduit.

The tank 60 in this example has a capacity of 1000 kg (=100%)

The control unit 600 used is a combination of a laptop and a WLAN router, with drivers for the provision unit 200, the measurement unit 300 and the distributor unit 400 (including fill level sensor 500) being installed on the laptop, and the wireless interfaces being WLAN interfaces. All WLAN interfaces are connected to the WLAN router. A digital user interface is also installed on the laptop, which gives a prompt for input of a target concentration for the tank, a target fill level for the tank, a tolerance range for the target concentration and a tolerance range for the target fill level, and gives the opportunity to select the corresponding values from a database. Also shown are an emergency stop button (PIN protected) and a start button.

The following settings are selected, assuming 100% concentration for the pure concentrate (expressed in % by weight; tolerances in absolute values), and the method is started. SK60 is the target concentration in tank 60, TK60 is the tolerance range around SK60, SF60 is the target fill level in tank 60, TF60 is the tolerance range around SF60, IK60 is the measured or calculated actual concentration in tank 60, IF60 is the measured actual fill level in tank 60; ΔF60 is the difference SF60-IF60; EK60 is the concentration of the fresh cooling lubricant emulsion required to attain the target concentration SK60 when topping up with the amount ΔF60.

| | Target | | Target | |
|---|---|---|---|---|
| | concentration | Tolerance | fill level | Tolerance |
| Tank 60 | SK60 = 10% | TK60 = 2% | SF60 = 90% | TF60 = 5% |

The control unit 600 is programmed to control the system 100 in such a way that a cascade of the following steps is performed, with the reference numerals corresponding to those in FIGS. 1, 3, 4, 7 and 8. Other cascades of steps are likewise conceivable. In particular, the flow rates can be varied in order to control the speed and accuracy of topping up.

First calibration of sensors 311 (refractometer), 312 (pH sensor), 313 (conductivity sensor):
1) close valve 410 to tank; open valve 408 to measurement unit 300; open valve 409;
2) open valve 211 to such an extent that fresh water flows at a rate of 9 kg/min (monitoring by turbine 210; if no flow is detected, stop method and issue error message); start pump 212 such that concentrate flows at a rate of 1 kg/min (monitoring by flowmeter 213; if no flow is detected, stop method and issue error message); continue conveying water and concentrate for 15 seconds (purge the sensors with fresh cooling lubricant emulsion at a concentration of SK60=10%); store the readings transmitted by the sensors 311, 312 and 313 to the control unit for 5 seconds; assign a concentration of SK60=10% to the received measured values using control unit 600;
3) repeat the steps listed under 2) at least for the following flow rate ratios of water to concentrate: 9.2 kg/min of water and 0.8 kg/min of concentrate, with assignment of the measured parameters to a concentration of SK60−TK60=8%;
8.8 kg/min of water and 1.2 kg/min of concentrate, with assignment of the measured parameters to a concentration of SK60+TK60=12%;
4) calculate a calibration curve that can be used to assign measured parameters of the cooling lubricant emulsion to a concentration.

First filling of tank 60:
5) calculate the concentration of the cooling lubricant emulsion (IK60) in tank 60 based on the amount of concentrate and water that has already flowed through (in the present case IK60=10%, since it is the average of 8%, 10% and 12%); measure the fill level with the fill level sensor 500 and transmit the value to control unit 600 (in the present case IF60=1%, since there was conveying at 10 kg/min for a total of 1 minute); calculate the difference ΔF60 from SF60 and IF60 (ΔF60=SF60−IF60=89%); calculate the required concentration EK60 of the fresh cooling lubricant emulsion required to attain the target concentration with the calculated fill level difference on attainment of SF60 (EK60=(SF60×SK60−IF60×IK60)/ΔF60=10%)
6) open valve 410 to tank 60; close valve 408 to measurement unit 300; open valve 211 to such an extent that fresh water flows at a rate of 90 kg/min (monitoring by turbine 210); start pump 212 such that concentrate flows at a rate of 10 kg/min (monitoring by flowmeter 213); convey until the fill level sensor 500 reports that the target fill level of SF60=90% has been attained (in the present case after about 8.9 minutes);
7) close valve 409; close valve 211; stop pump 212.

Measurement of the at least one parameter and conditioning:
8) open valve 408 to measurement unit 300; start pump 50 (transfer of cooling lubricant emulsion from tank 60 into measurement unit 300); continuously measure the at least one parameter with sensors 311, 312 and 313, and transmit the measured values to the control unit; continuously measure the fill level in tank 60 with the fill level sensor 500 and transmit the reading to control unit 600; if IF60 falls below SF60−TF60=85%, or IK60 falls below SK60−TK60=8%, or rises above SK60+TK60=12%, go to step 9);
9) calculate the difference ΔF60=SF60-IF60; calculate the required concentration EK60=(SF60×SK60−IF60×IK60)/ΔF60 required to attain the target concentration SK60 when topping up with the required amount ΔF60 of fresh cooling lubricant emulsion.
10) stop pump 50; close valve 408 to measurement unit 300; open valve 409; open valve 211 such an extent that fresh water flows at a rate of (100%−EK60)×100 kg/min (monitoring by turbine 210); start pump 212 such that concentrate flows at a rate of EK60×100 kg/min (monitoring by flowmeter 213); convey the fresh cooling lubricant emulsion until the fill level sensor 500 reports that the target fill level of SF60=90% has been attained (time about (ΔF60/100 kg) min);
11) close valve 409; close valve 211; stop pump 212; go to step 8).

### Recalibration:

12) if repetition of step 8) after performance of step 11) does not attain SK60: stop pump 50; close valve 410 to tank; open valve 409; repeat step 2) of the first calibration; adjust the calibration curve using the new measurements for SK60; go to step 8).

The invention is not limited to the working examples described herein and the aspects highlighted therein. Instead, within the scope specified by the claims, a large number of modifications are possible within the scope of activity of the person skilled in the art.

### LIST OF REFERENCE NUMERALS

10, 10′ machine tool
40 filter basket
50 pump
60,60′ tank
70 concentrate vessel
80 power cable
90 data cable (with power supply)
100 system of the invention
200 provision unit
201, 301, 401 housings
202, 302 AC power connection
203, 303 AC-DC converter
204, 304 wireless interface
205-207, 221, 223, 306, 307, 403-407, 403′, 416-418
208 static mixer
209, 214, 310, 424 check valve
210, 414 turbine
liquid connection
211, 408, 409, 410, 425, 426 valve
212, 422 volumetric gear pump
213, 423 oval gear flowmeter
215 fill level sensor for concentrate vessel, fresh water vessel or additive vessel;
216-218, 222, 308, 309, 411, 412, 419-421 liquid conduit
219, 413 pressure sensor
220 compressed air pump
224 compressed air conduit
300, 300′ measurement unit
305, 402 interface for data cables
311-313 sensor
400, 400′ distributor unit
415 three-way valve
500 fill level sensor
600 control unit
700 conduit with free end
701 free end
702 identification feature

1-16. (canceled)

17. A system for conditioning a cooling lubricant emulsion for machine tools comprising:
a measurement unit comprising at least one sensor for determining at least one parameter of the cooling lubricant emulsion;
a provision unit for providing fresh cooling lubricant emulsion, wherein the provision unit is configured to mix a defined amount of a concentrate with a defined amount of water and to provide the mixture as a fresh cooling lubricant emulsion;
a fill level sensor configured to measure the fill level in a tank;
a distributor unit configured to be connected to the measurement unit, the provision unit, and the tank for controlling an exchange of the cooling lubricant emulsion between the measurement unit, the provision unit, and the tank; and
a control unit configured to control and exchange data with the provision unit, the measurement unit, the distributor unit, and the fill level sensor,
wherein the provision unit is spatially separated from the measurement unit and is not in direct liquid exchange with the measurement unit.

18. The system of claim 17,
wherein the provision unit has a housing, and
wherein the measurement unit and the distributor unit have a common housing.

19. The system of claim 17, wherein the control unit is configured to:
a) actuate the provision unit to mix the defined amount of the concentrate with the defined amount of water and to provide the mixture as the fresh cooling lubricant emulsion;
b) actuate the distributor unit to guide the fresh cooling lubricant emulsion from the provision unit into the measurement unit or into the tank, or to guide the cooling lubricant emulsion from the tank into the measurement unit, and/or to optionally guide at least a portion of the cooling lubricant emulsion from the measurement unit into the tank;
c) receive measurement data from at least one sensor of the measurement unit and the fill level sensor;
d) calculate a top-up amount required to convert the fill level in the tank from a measured actual fill level to a defined target fill level;
e) calculate a top-up concentration of the fresh cooling lubricant emulsion required for a calculated top-up amount to convert a measured actual concentration in the tank to a defined target concentration; and
f) calibrate the at least one sensor of the measurement unit.

20. The system of claim 17, wherein:
the fill level sensor is incorporated into the distributor unit, and
the distributor unit is configured to be secured to the tank to measure the fill level in the tank.

21. The system of claim 17, further comprising:
a conduit for the fresh cooling lubricant emulsion for enabling a reversible connection between the provision unit and the distributor unit,
wherein the conduit has at least one free end,
wherein the at least one free end has an identification feature uniquely assigned to the conduit,
wherein the at least one free end is configured to be reversibly connected to the provision unit, the distributor unit, or both, and
wherein the distributor unit, the provision unit, or both are configured to recognize the identification feature on establishment of a connection with the at least one free end.

22. The system of claim 21, wherein the identification feature uniquely assigned to the conduit is a radio-frequency identification (RFID) transponder.

23. The system of claim 21, wherein the at least one free end is configured to be reversibly connected to the provision unit, the distributor unit, or both via a quick-release fastener.

24. The system of claim 17, further comprising:
at least one further measurement unit;
at least one further distributor unit; and
at least one further fill level sensor,
wherein the at least one further fill level sensor is configured to measure the fill level in the tank,
wherein the provision unit is spatially separated from the at least one further measurement unit and is not in direct liquid exchange with the at least one further measurement unit,
wherein the at least one further distributor unit is configured to be connected to the at least one further measurement unit, the provision unit, and the tank and configured to control the exchange of the cooling lubricant emulsion between the at least one further measurement unit, the provision unit, and the tank,
wherein the system is configured to condition the cooling lubricant emulsion in at least two tanks, and
wherein the control unit is configured to determine which distributor unit the fresh cooling lubricant emulsion from the provision unit is guided through.

25. The system of claim 17, wherein:
the provision unit comprises a first wireless interface, and
the control unit is configured to communicate with the provision unit via the first wireless interface.

26. The system of claim 25, wherein:
the measurement unit comprises a second wireless interface, and
the control unit is configured to communicate with the measurement unit via the second wireless interface.

27. The system of claim 17, wherein:
the distributor unit is configured to be connected to the measurement unit via a data cable interface, and
the control unit is configured to communicate with the distributor unit via the measurement unit and the data cable interface.

28. The system of claim 17, wherein the at least one parameter comprises a parameter selected from refractive index, pH, transmittance, reflectance, oil content, and conductivity of the cooling lubricant emulsion.

29. The system of claim 17, wherein the provision unit is configured to be reversibly connected to the distributor unit and to be moved at least in a separated state independently of the measurement unit, the distributor unit, and the tank.

30. The system of claim 17, wherein:
the provision unit is connected to and in liquid exchange with the distributor unit via a first conduit,
the measurement unit is connected to and in liquid exchange with the distributor unit via at least one second conduit,
the distributor unit is in liquid exchange with the tank via at least one third conduit, and
the control unit has a data connection with the provision unit, the measurement unit, and the distributor unit.

31. A method for conditioning a cooling lubricant emulsion for machine tools, the method comprising:
providing a cooling lubricant emulsion in a tank;
withdrawing the cooling lubricant emulsion from the tank;
detecting at least one parameter of the cooling lubricant emulsion with at least one sensor;
determining a concentration of the cooling lubricant emulsion in the tank using the at least one parameter to determine;
returning at least a portion of the cooling lubricant emulsion to the tank;
measuring a fill level in the tank using a fill level sensor;
calculating a top-up concentration and a top-up amount;
providing fresh cooling lubricant emulsion having the calculated top-up concentration in the calculated top-up amount;
calibrating the at least one sensor by purging the at least one sensor; and
performing a measurement with the fresh cooling lubricant emulsion having a defined ratio of a concentrate to water,
wherein the fresh cooling lubricant emulsion is provided by a provision unit,
wherein the at least one sensor is part of a measurement unit,
wherein the provision unit is spatially separated from the measurement unit and is not in direct liquid exchange with the measurement unit,
wherein the provision unit, the measurement unit, and the tank are connected to a distributor unit configured to control an exchange of the cooling lubricant emulsion between the provision unit, the measurement unit, and the tank, and
wherein the method is controlled by a control unit.

32. The method of claim 31, wherein the method is performed with a system for conditioning a cooling lubricant emulsion for machine tools, the system comprising:
the measurement unit comprising the at least one sensor for determining the at least one parameter of the cooling lubricant emulsion;
the provision unit for providing the fresh cooling lubricant emulsion, wherein the provision unit is configured to mix a defined amount of the concentrate with a defined amount of water and to provide the mixture as the fresh cooling lubricant emulsion;
the fill level sensor configured to measure the fill level in the tank;
the distributor unit configured to be connected to the measurement unit, the provision unit, and the tank and configured to control the exchange of the cooling lubricant emulsion between the measurement unit, the provision unit, and the tank; and
the control unit configured to control and exchange data with the provision unit, the measurement unit, the distributor unit, and the fill level sensor,
wherein the provision unit is spatially separated from the measurement unit and is not in direct liquid exchange with the measurement unit.

33. The method of claim 31, further comprising:
providing the cooling lubricant emulsion for each of at least two machine tools in a respective tank, wherein each of the respective tanks is connected to a respective distributor unit and wherein each of the respective distributor units is connected to a respective measurement unit;
providing the fresh cooling lubricant emulsion in a common provision unit; and
controlling the exchange of the cooling lubricant emulsion between the provision unit and the respective distributor units manually by changing the distributor unit connected to the provision unit or automatically by the control unit.

34. The method of claim 31, further comprising:
providing the fresh cooling lubricant emulsion having the defined ratio of the concentrate to water in the provision unit;
introducing the fresh cooling lubricant emulsion from the provision unit into the measurement unit with the distributor unit;
calibrating the at least one sensor with the fresh cooling lubricant emulsion directed into the measurement unit by performing a measurement with the fresh cooling lubricant emulsion having the defined ratio of the concentrate to water, and optionally conducting the fresh cooling lubricant emulsion at least partly into the tank after the measurement;
withdrawing the cooling lubricant emulsion from the tank and introducing the cooling lubricant emulsion into the measurement unit;
measuring the at least one parameter of the cooling lubricant emulsion with the at least one sensor in order to ascertain an actual concentration of the cooling lubricant emulsion in the tank with reference to the at least one parameter, and optionally returning the cooling lubricant emulsion at least partly to the tank;
measuring the fill level in the tank with the fill level sensor;
calculating the top-up amount required to attain a defined fill level in the tank;
calculating the top-up concentration required for the calculated top-up amount to condition the concentration of the cooling lubricant emulsion in the tank to a fixed target value;
providing the fresh cooling lubricant emulsion having the calculated required top-up concentration in the calculated top-up amount in the provision unit; and
introducing the fresh cooling lubricant emulsion into the tank with the distributor unit in order to condition the cooling lubricant emulsion in the tank.

35. The method of claim 31,
wherein a target concentration of the cooling lubricant emulsion in the tank, a tolerance range for a concentration around the target concentration, a target fill level in the tank, and a tolerance range for the fill level around the target fill level are defined, and
wherein the control unit performs at least the following steps:
i) sending a command to the provision unit to commence providing of fresh cooling lubricant emulsion with a ratio of concentrate to water defined for a calibration;
ii) sending a command to the distributor unit to commence introducing of the cooling lubricant emulsion provided by the provision unit into the measurement unit, wherein the cooling lubricant emulsion from the measurement unit is optionally directed at least partly into the tank;
iii) sending a command to the measurement unit to perform a measurement of at least one parameter with the at least one sensor and to transmit the measurement data to the control unit in order to calibrate the at least one sensor;
iv) sending a command to the provision unit to stop the provision of the fresh cooling lubricant emulsion;
v) sending a command to the distributor unit to stop the introduction of the cooling lubricant emulsion provided by the provision unit into the measurement unit;
vi) sending a command to the distributor unit to commence with withdrawal of the cooling lubricant emulsion from the tank and introduction of the cooling lubricant emulsion into the measurement unit, and returning of the cooling lubricant emulsion from the measurement unit at least partly into the tank;
vii) sending a command to the measurement unit to perform a measurement of the at least one parameter with the at least one sensor and to transmit the measurement data to the control unit in order to determine the actual concentration of the cooling lubricant emulsion;
viii) sending a command to the distributor unit to stop the withdrawal of the cooling lubricant emulsion from the tank and introduction of the cooling lubricant emulsion into the measurement unit;
ix) sending a command to the fill level sensor to measure the fill level in the tank and to transmit the measurement data to the control unit to determine the actual fill level in the tank;
x) checking whether the ascertained actual fill level and the ascertained actual concentration are within the defined tolerance ranges; if so, repeating steps vi) to x), and if not, continuing from step xi);
xi) calculating the required top-up amount from the difference between the defined target fill level and the ascertained actual fill level and calculating the top-up concentration required to achieve the target concentration with the calculated top-up amount based on the actual concentration;
xii) sending a command to the provision unit to commence providing of fresh cooling lubricant emulsion with the calculated top-up concentration;
xiii) sending a command to the distributor unit to commence introducing of the cooling lubricant emulsion provided by the provision unit into the tank to condition the cooling lubricant emulsion in the tank;
xiv) sending a command to the provision unit, when the target fill level has been attained or the calculated top-up amount has been provided, to stop the provision of fresh cooling lubricant emulsion; and
xv) sending a command to the distributor unit to stop the introduction of the cooling lubricant emulsion provided by the provision unit into the tank.

36. The method of claim 31, wherein the at least one parameter comprises a parameter selected from refractive index, pH, transmittance, reflectance, oil content, and conductivity of the cooling lubricant emulsion.
